(19) United States
(12) Patent Application Publication
(10) Pub. No.: US 2017/0096703 A1
(43) Pub. Date: Apr. 6, 2017

(54) ANALYSIS OF A POLYMER FROM MULTI-DIMENSIONAL MEASUREMENTS

(71) Applicant: Oxford Nanopore Technologies Ltd., Oxford (GB)

(72) Inventors: Kevin Thomas Dolan, Oxford (GB); Christopher James Wright, Oxford (GB)

(73) Assignee: Oxford Nanopore Technologies Ltd., Oxford (GB)

(21) Appl. No.: 15/127,885

(22) PCT Filed: Mar. 17, 2015
(86) PCT No.: PCT/GB2015/050776

§ 371 Date: Sep. 21, 2016

(30) Foreign Application Priority Data
Mar. 21, 2014 (GB) 1405090.0
Oct. 16, 2014 (GB) 1418373.5

(51) Int. Cl. C12Q 1/68; G06F 19/22; G06F 19/24; G01N 33/487

(52) U.S. Cl. CPC C12Q 1/6869; G01N 33/48721; G06F 19/22; G06F 19/24

(57) ABSTRACT

A target sequence of polymer units is estimated from plural series of measurements taken from sequences of polymer units that comprise the target sequence or a complementary sequence. Each measurement is dependent on a k-mer (k polymer units). Models treat the measurements as observations of k-mer states, comprising transition weightings in respect of transitions between successive k-mer states and emission weightings for different measurements being observed. An estimated alignment mapping between the plural series of measurements is derived based on an application of the models to each series. An estimate of the target sequence of polymer units is generated by applying the models, treating the types of k-mer state of each model and the measurements as dimensions of a plural dimensional k-mer state and plural dimensional observations. Constraint of paths through the plural dimensional k-mer states using the derived alignment mapping greatly reduces the required processing.

The present invention relates to the generation of an estimate of a target sequence of polymer units in a polymer, for example but without limitation a polynucleotide, from measurements taken from polymers.

By way of definition, a group of k polymer units is hereinafter referred to as a k-mer. In general, k can take the value one, in which case a k-mer is a single polymer unit or can be a plural integer. Each given polymer unit may be of different types, depending on the nature of the polymer. For example, in the case that the polymer is a polynucleotide, the polymer units are nucleotides and the different types are nucleotides including different nucleobases (such as cytosine, guanine, etc.). Each given k-mer may therefore also be of different types, corresponding to different combinations of the different types of each polymer unit of the k-mer.

There are many types of measurement system that provide measurements of polymer units for the purpose of determining the sequence. For example but without limitation, one type of measurement system uses a nanopore through which the polymer is translocated. Some property of the system depends on the polymer units in the nanopore, and measurements of that property are taken. This type of measurement system using a nanopore has considerable promise, particularly in the field of sequencing a polynucleotide such as DNA or RNA, and has been the subject of much recent development.

Such nanopore measurement systems can provide long continuous reads of polynucleotides ranging from hundreds to tens of thousands (and potentially more) nucleotides. The data gathered in this way comprises measurements, such as measurements of ion current, where each translocation of the sequence through the sensitive part of the nanopore results in a slight change in the measured property.

In practical types of the measurement system, it is difficult to provide measurements that are dependent on a single polymer unit, and instead the value of each measurement is dependent on a k-mer, where k is a plural integer. Conceptually, this might be thought of as the measurement system having a “blunt reader head” that is bigger than the polymer unit being measured. In such a situation, the number of different k-mers to be resolved increases to the power of k. When measurements are dependent on large numbers of polymer units (large values of k), measurements taken from k-mers of different types can be difficult to resolve, because they provide signal distributions that overlap, especially when noise and/or artefacts in the measurement system are considered. This is to the detriment of estimating the underlying sequence of polymer units.

Where k is a plural integer, it is possible to combine information from multiple measurements of overlapping k-mers that each depend in part on the same polymer unit to obtain a single value that is resolved at the level of a polymer unit. By way of example, WO-2013/041878 discloses a method of estimating a sequence of polymer units in a polymer from at least one series of measurements related to the polymer that makes use of a model in respect of each series of measurements that treats the measurements as observations of a series of k-mer states of different possible types. The model comprises: transition weightings, in respect of each transition between successive k-mer states in the series of k-mer states, for possible transitions between the possible types of k-mer state; and emission weightings, in respect of each type of k-mer state that represent the chances of observing given values of measurements for that k-mer. The model may be for example a Hidden Markov Model. Such a model can improve the accuracy of the estimation by taking plural measurements into account in the consideration of the likelihood predicted by the model of the series of measurements being produced by sequences of polymer units.

In order to improve the accuracy over that achievable from a single series of measurements, it is possible to make use of plural series of measurements taken from respective sequences of polymer units that correspond to the target sequence by comprising the target sequence or a sequence having a predetermined relationship with the target sequence, for example being a complementary sequence. These may be considered as multi-dimensional measurements, each series of measurements providing a dimension of the information. Multi-dimensional measurements may be used to improve accuracy of estimation. As well as reducing errors by simply increasing the number of measurements obtained, there may be an additional gain in that the errors associated with each series of measurements may tend to be not systematic, such that different useful information can be obtained from each series.

By way of example, WO-2013/041878 discloses that the method of estimating a sequence of polymer units disclosed therein may be applied to multi-dimensional measurements by applying a multi-dimensional model. Whilst that approach is feasible taught in WO-2013/041878, it requires a large amount of processing power when the measurements of different series are not registered, because it is necessary to perform an analysis that takes account of all possible alignments between the plural series of measurements. Compared to the analysis performed for a single series of measurements in one dimension which approximately scales with the number of measurements, the processing required to consider all possible all possible paths through the plural dimensional k-mer states approximately scales with the number of measurements to the power of the number of series.

The techniques required to analyse long series of measurements, for example as achievable using nanopore measurement systems, are intrinsically intensive and processing power is limited by practical considerations. Such practical limitations on processing have an impact on the accuracy of the estimate that it is possible to achieve from a given set of plural series of measurements, especially as the number of series and/or measurements in each series is increased.

Long reads from single DNA molecules have several significant advantages over the short reads produced by the current “second” generation of sequencing machines, three examples include: assembling genomes, assigning Single Nucleotide Polymorphisms (SNPs) and variants to the maternal or paternal chromosomes, and accessing repetitive regions of the genome. Long reads may be considered as measurements of an unfragmented sequence of DNA polymer units which is greater than about 2 kB (2 kilobases) in length.

The copying of genomic regions, and genes in particular, has been an important mechanism in the evolution of new function in genomes (leading to the creation of “paralogous” genes with similar but specialized functions, for example) and means there are regions of very similar sequence for which short reads are ambiguous. In the extreme case, there are copy number variants of identical genes such as the CCL3L1 gene involved in HIV resistance. The ambiguity of mapping short reads to a reference genome in these regions means that simple questions like how many repeats occur where are difficult, whereas such questions may be trivially answered by a long read which spans the entire region. Repeated sequence also arises in the form of microsatellites, where a simple pattern of sequence is repeated, and the number of repeats is a characteristic of genetic diseases like Huntington's.

Repeats of sequence such as those described above and other similarities cause problem when attempting to assemble a genome from multiple short reads. The result of assembly tends to consist of many fragments of contiguous sequence “contigs”, whose order is ambiguous, rather than a single fully resolved chromosome—long reads can easily span across contigs, past the ambiguous sequence, and so resolve the order.

SNPs may occur on either maternal or paternal chromosome but whether two SNPs occur on the same chromosome is often critical in disease, for example does a person have two copies of a gene containing errors or one good and one bad copy? A long read, which may cover the entire gene, is much more likely than a short read to span multiple SNPs and so prove they are on the same or different chromosomes (“phased”); by chaining multiple such reads together, even distant SNPs can be phased.

Thus it would be highly desirable to be able to determine long sequences and to be able to do so without an undue processing burden when applying such multi-dimensional techniques.

According to an aspect of the present invention, there is provided a method of generating an estimate of a target sequence of polymer units from plural series of measurements taken from respective measured sequences of polymer units in the same or different polymers, wherein the respective measured sequences correspond to the target sequence by comprising the target sequence or a sequence having a predetermined relationship with the target sequence, each measurement being dependent on a k-mer, being k polymer units of the respective sequence of polymer units, where k is an integer,

the method using a model in respect of each series of measurements that treat the measurements as observations of a series of k-mer states of different possible types, and comprises:

in respect of each transition between successive k-mer states in the series of k-mer states, transition weightings for possible transitions between the possible types of the k-mer states; and

in respect of each type of k-mer state, emission weightings for different measurements being observed when the k-mer state is of that type,

the method comprising:

deriving an estimated alignment mapping between the plural series of measurements; and

generating an estimate of the target sequence of polymer units from the plural series of measurements by applying the models in a manner that treats the types of k-mer state of each model as dimensions of a plural dimensional k-mer state and treats the measurements of each series as plural dimensional observations of those plural dimensional k-mer states, and that paths through the plural dimensional k-mer states are constrained using the derived alignment mapping between the plural series of measurements.

Thus, the method obtains the power of using plural series of measurements taken from respective sequences of polymer units that correspond to the target sequence. Such a sequence of polymer units may comprise the target sequence or may comprise a sequence having a predetermined relationship with the target sequence, for example being a complementary sequence. Thus, measurements from each series may contribute information to the estimate of the target sequence.

Further, the method makes use of a model in respect of each series of measurements that treats the measurements as observations of a series of k-mer states of different possible types, for example a model of the type disclosed in WO-2013/041878, typically a Hidden Markov Model. This allows the predictive power of such a model to be harnessed in providing the estimate of the target sequence.

However, the method does not rely on generating estimates of the target sequence of polymer units from each of the series of measurements and then seeking to align those estimates of the target sequence in the polymer unit space. Hereinafter, such a technique will be referred to as a 1D polymer unit technique, because separate estimates of the polymer units are estimated (called), each from a single series of measurements. The method can provide a significant improvement accuracy in estimation over a 1D polymer unit technique, even when the measurement technique used to take the measurements has levels of error in the individual measurements that are worse than those required to align the estimates of the target sequence of polymer units derived from the different series of measurements.

Instead, the method derives an estimated alignment mapping between the measurements of the respective series. The estimated alignment mapping between the measurements of the respective series is then used in the generation of estimate of the target sequence of polymer units. In particular, the models are applied in a manner that treats the types of k-mer state of each model as dimensions of a plural dimensional k-mer state and treats the measurements of each series as plural dimensional observations of those plural dimensional k-mer states. That plural dimensional approach increases accuracy by combining information from all homologous features, However, the estimated alignment mapping is used to constrain the paths through the plural dimensional k-mer states. This reduces the scope of the analysis to a small portion of the total state-space, greatly reducing both computational complexity and memory requirements, and hence the amount of processing power required. Although the processing power is of course increased above that for a single series of measurements in one dimension, it is significantly less than an unconstrained analysis in plural dimensions. In particular, the required processing power scales with the number of measurements, rather than the number of measurements to the power of the number of series. Thus the method is particularly advantageous for determining long reads, for example of sequences of 2000 polymer units or greater. However the method is equally applicable to determining sequences shorter than 2000 polymer units.

Furthermore, in the face of practical limitations on processing power that may be employed, the reduction in the required processing power is sufficient to improve the accuracy of estimation that may be achieved with the available processing resource.

There are several different techniques for deriving an estimated alignment mapping between the measurements of the respective series. Some techniques may use models that treat the measurements as observations of a series of k-mer states of different possible types, comprising: transition weightings for transitions between successive k-mer states in the series of k-mer states; and in respect of each k-mer state emission weightings for different measurements being observed. In general terms, this allows the alignment mapping to be derived in the space of the k-mer states, rather than in the space of the polymer units. This allows the estimation of the alignment mapping to preserve much of the information contained in the original measurements, while at the same time abstracting the sequences from the measurement space to the k-mer space that allows for direct correspondence of data. Since the k-mer states can be informed by every measurement in the plural series, they can be much more informative about the type of k-mer state of each k-mer in the target sequence than the corresponding raw measurement.

There will now be described some non-limitative examples of techniques for basing derivation of the estimated alignment mapping on the models.

In a first type of technique, the estimated alignment mapping is derived by:

generating, in respect of each series of measurements, a series of estimates of k-mer states by applying the model of the series of measurements to the series of measurements; and

deriving the estimated alignment mapping between the series of measurements by comparing the plural series of estimates of the k-mer states.

This technique is “symmetrical” in the sense that each series of measurements is processed in the same manner to derive a series of estimates of k-mer states. The alignment mapping is estimated from those series of estimates of k-mer states and hence derived in the space of the k-mer states.

The series of estimates of k-mer states that is generated and compared may take a variety of forms.

In a first form, each estimate of a k-mer state may comprise weightings for each possible type of k-mer state. In that case, the series of estimates of k-mer states may also comprises an estimate of a k-mer state in respect of each measurement, i.e. with a one-to-one mapping between the estimates of a k-mer state and the measurements. As each estimate of a k-mer state may comprises weightings for each possible type of k-mer state, each estimate contains information on all possible types of k-mer state, not just the single most likely k-mer state for a given measurement. This can provide for accuracy in the alignment mapping, particularly helping with errors associated with movement of the polymer in the measurement system. For example, in the event of a missing event, by comparison a 1D polymer unit technique might estimate that the most likely sequence is one with a polymer unit missing, but the present method can take account of weightings for different types of k-mer state that may still have substantial weight associated with the correct k-mer state, allowing the alignment to be correctly estimated.

In a second form, each estimate of a k-mer state may comprise an estimated k-mer state.

With this form of the estimates, each series of estimates of the k-mer states may comprise an estimate of a k-mer state in respect of each measurement. That may be considered as making a call of the k-mer state in respect of each measurement. These estimates may not be mapped properly to the underlying sequence of polymer units, for example if more than one measurement is dependent on the same k-mer (a “stay”), or if no measurement is taken from a given k-mer (a “skip”), as may occur in the case of errors associated with improper movement of the polymer in the measurement system. However, in this case there is a one-to-one mapping between the estimates of a k-mer state and the measurements.

Alternatively, with this form of the estimates as an estimated k-mer state, each series of estimates of the k-mer states may comprises an estimated k-mer state in respect of each k-mer in an underlying sequence that corresponds with an estimate of the measured sequence of polymer units. In this case, the estimates are mapped to an estimate of underlying sequence of polymer units, so this may be considered as making a call of the k-mer states corresponding to the measured sequence, i.e. a one-dimensional calls from each series of measurements. In order to derive the estimated alignment mapping from the estimates, then the estimated k-mer states in respect of each k-mer in the underlying sequence may be mapped to the measurements.

Such estimated k-mer states in respect of each k-mer in an underlying sequence may be derived by generating an initial series of estimates of k-mer states in respect of each measurement by applying the model of the series of measurements to the series of measurements, and then analysing the initial series of estimates of k-mer states in respect of each measurement to derive the estimated k-mer state in respect of each k-mer in said underlying sequence.

Despite the similarities between the above-described forms, it has been found in some circumstances that the second form can yield a more accurate estimated alignment mapping between the event sequences than can the first.

In a second type of technique, the estimated alignment mapping is derived by:

in respect of a first one of the series of measurements, performing the steps of:

generating an initial series of estimates of k-mer states in respect of each measurement by applying the model of the first series of measurements to the first series of measurements; and

analysing the initial series of estimates of k-mer states in respect of each measurement to derive an estimated k-mer state in respect of each k-mer in an underlying sequence that corresponds with an estimate of the measured sequence of polymer units, and

deriving an estimated alignment mapping between the plural series of measurements by applying the model of the other series of measurements with modified transition weightings corresponding to the derived series of estimated k-mer states in respect of each k-mer in an underlying sequence.

This technique is “asymmetrical” in the sense that each series of measurements is processed in a different manner. The first series of measurements is processed to derive mapped to an estimate of underlying sequence of polymer units. That may be considered as making a call of the k-mer states corresponding to the measured sequence based on solely the first series of measurements, i.e. a one-dimensional call from a single series of estimates. The alignment mapping is estimated by using the estimated k-mer states in respect of each k-mer in an underlying sequence to provide modified transition weightings for the model of the other series of measurements. Application the model of the other series of measurements with such modified transition weightings provides an estimated alignment mapping between the plural series of measurements, because it effectively fits the other series of measurements to the estimated k-mer states of the underlying sequence estimated from the first series of measurements. Despite the asymmetrical nature of the technique, the estimated alignment mapping is nonetheless derived in the space of the k-mer states, because it is based on application of the models in respect of each series of measurements

Based on application to some typical series of measurements, it is found that the second type of technique provides, in some circumstances, a more accurate alignment mapping, at the expense of slight additional computation effort, than the first form of the first type of technique. This is somewhat surprising given the asymmetric nature of the second type of technique.

In respect of all techniques, a more accurate alignment mapping may reduce the processing requirements required to generate the estimate of the target sequence of polymer units from the plural series of measurements by allowing the constraint on the paths through the plural dimensional k-mer states to be tighter. That in turn may improve the accuracy of estimation of the target sequence of polymer units when restricted by practical limitations on processing power.

The most appropriate technique in the sense of providing the most accurate estimated alignment mapping may not necessarily be known or estimated in advance and may depend upon various experimental factors such as the ambient temperature and the k-mer type. However, for a given type of measurement systems and/or sample, the various techniques may be easily evaluated with respect to each other, for example by applying the techniques with respect to the measurement of known sequences under defined experimental conditions.

According to other aspects of the present invention, there is provided an analysis system that implements a method similar to that of the first aspect of the invention.

To allow better understanding, embodiments of the present invention will now be described by way of non-limitative example with reference to the accompanying drawings, in which:

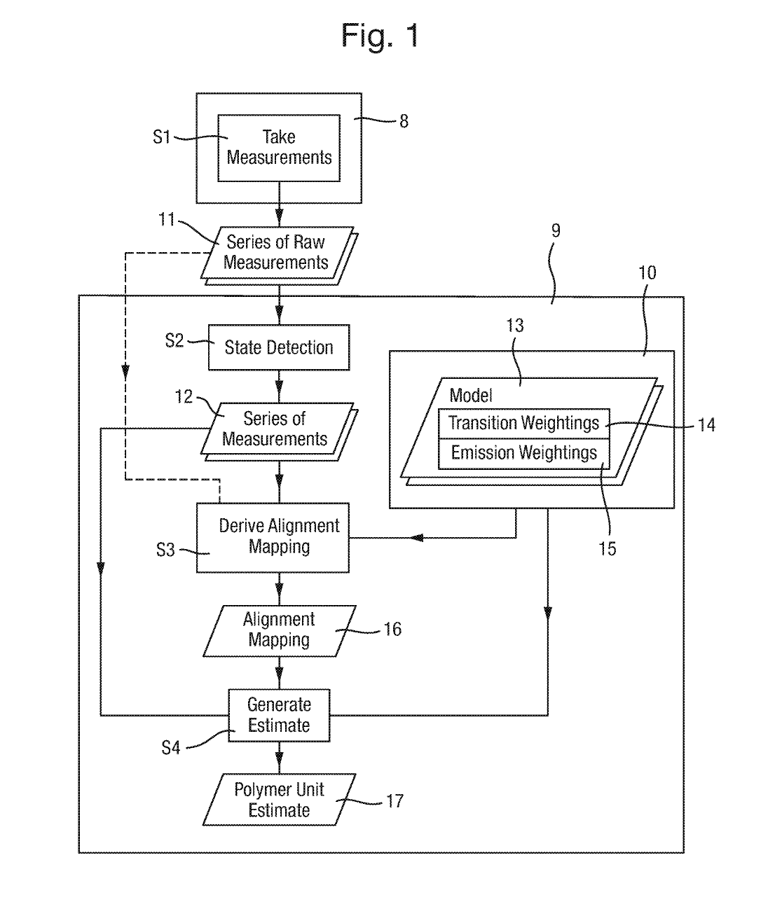

FIG. 1 is a flowchart of a method of generating an estimate of a target sequence of polymer units from plural series of measurements;

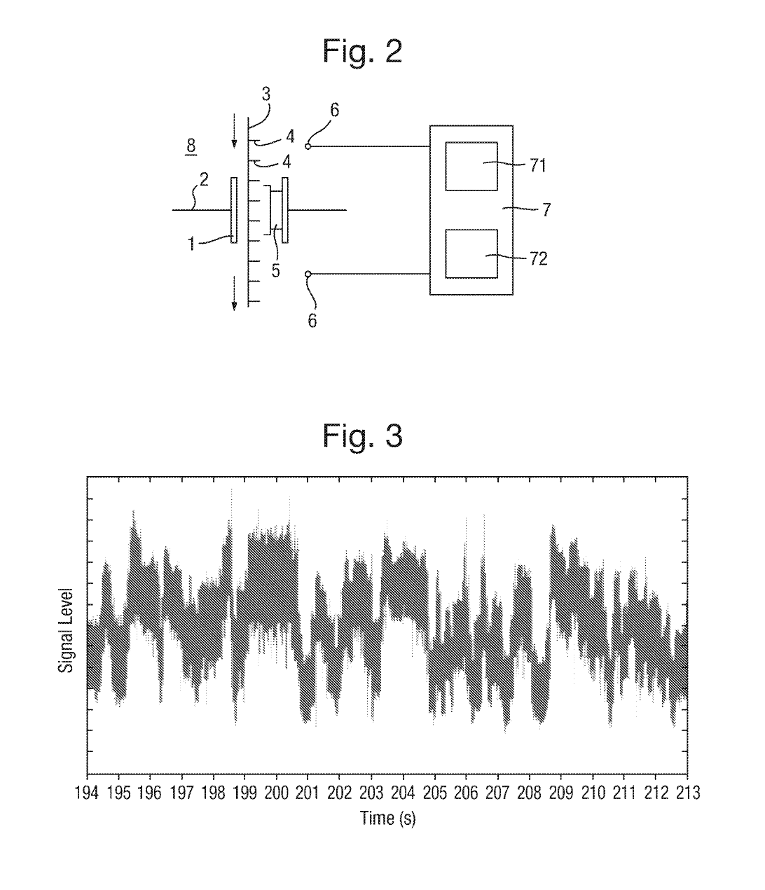

FIG. 2 is a schematic diagram of a measurement system comprising a nanopore;

FIG. 3 is a plot of a signal of an event measured over time by a measurement system;

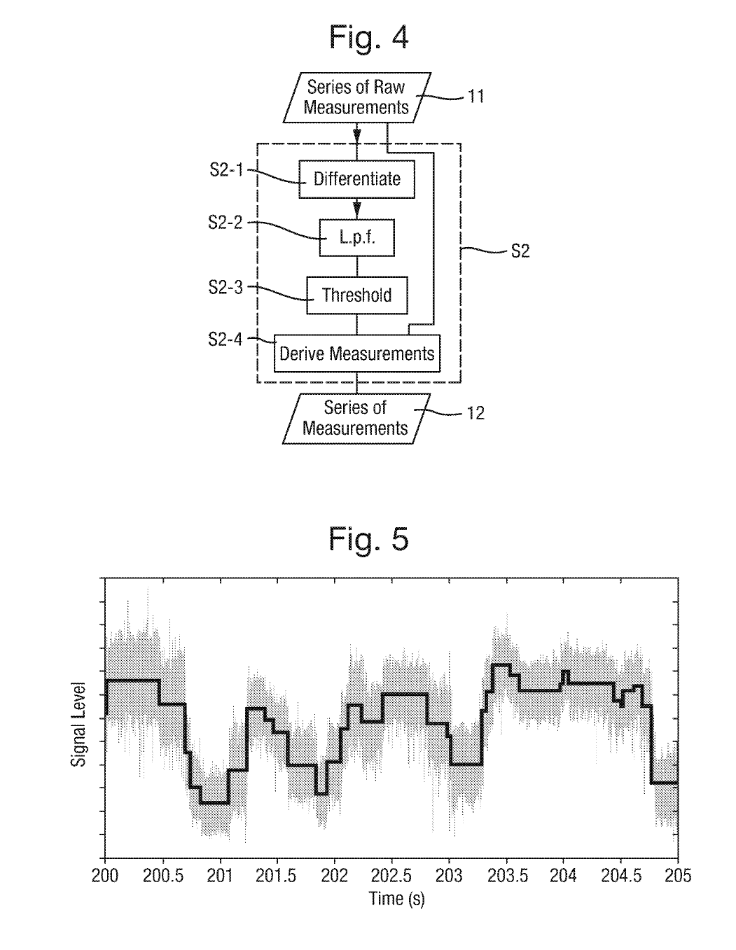

FIG. 4 is a flowchart of a state detection step of FIG. 1;

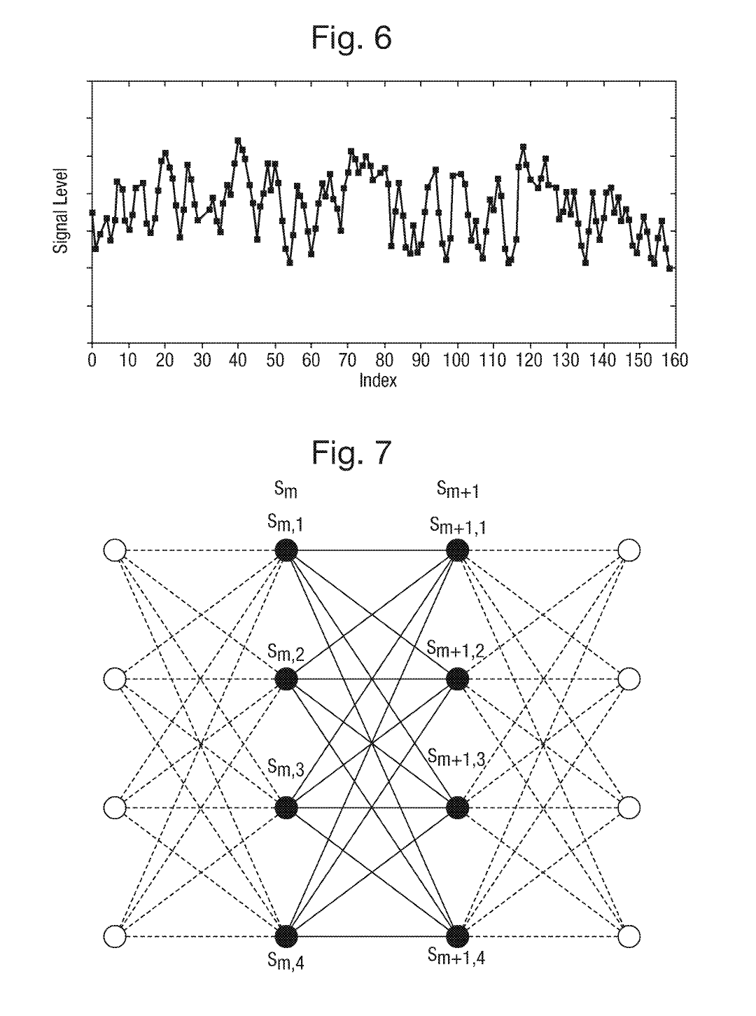

FIGS. 5 and 6 are plots, respectively, of a series of raw measurements subject to the state detection step and of the resultant series of measurements;

FIG. 7 is a pictorial representation of a transition matrix;

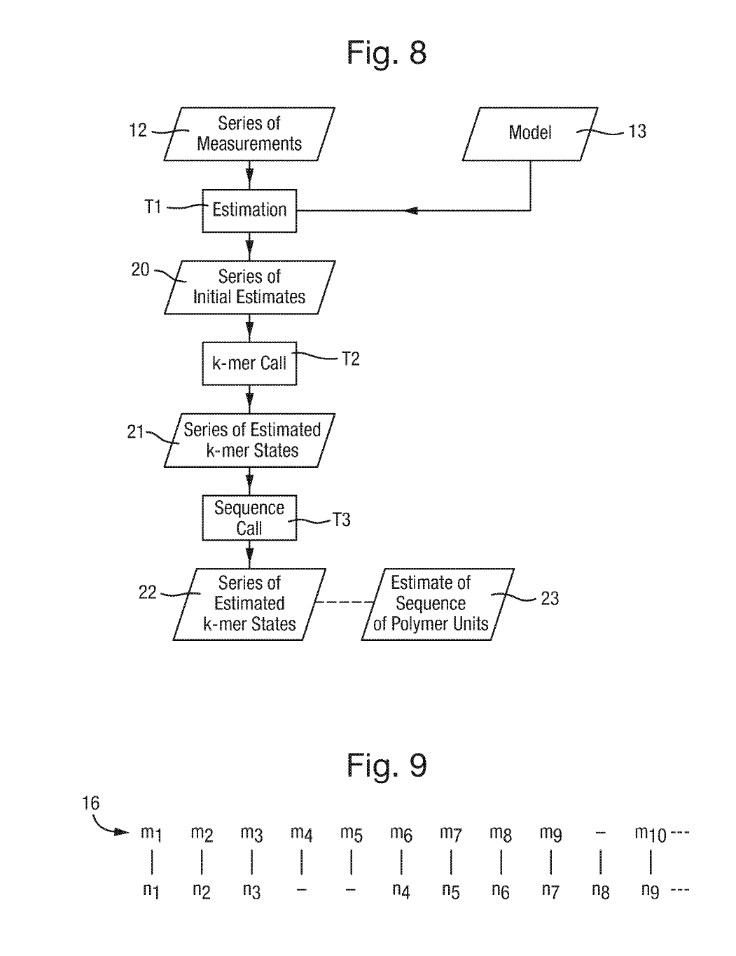

FIG. 8 is a flowchart of a method of generating an estimate of a target sequence of polymer units from a single series of measurements;

FIG. 9 illustrates an alignment between two series of measurements;

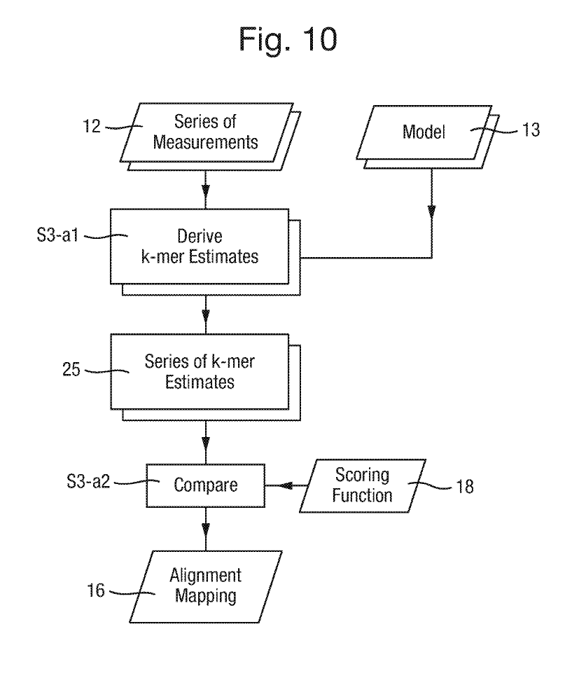

FIG. 10 is a flow chart of a first method of performing an alignment step of FIG. 1;

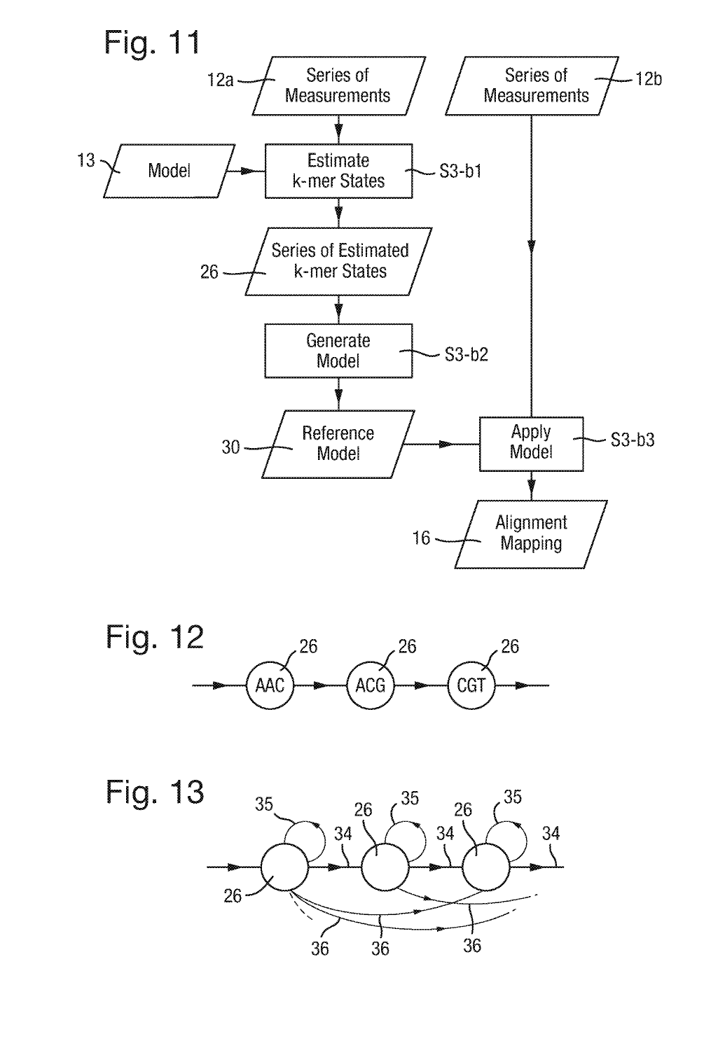

FIG. 11 is a flow chart of a second method of performing an alignment step of FIG. 1;

FIG. 12 is a state diagram of an example of a reference series of k-mer states;

FIG. 13 is a state diagram of a reference series of k-mer states illustrating possible types of transition between the k-mer states;

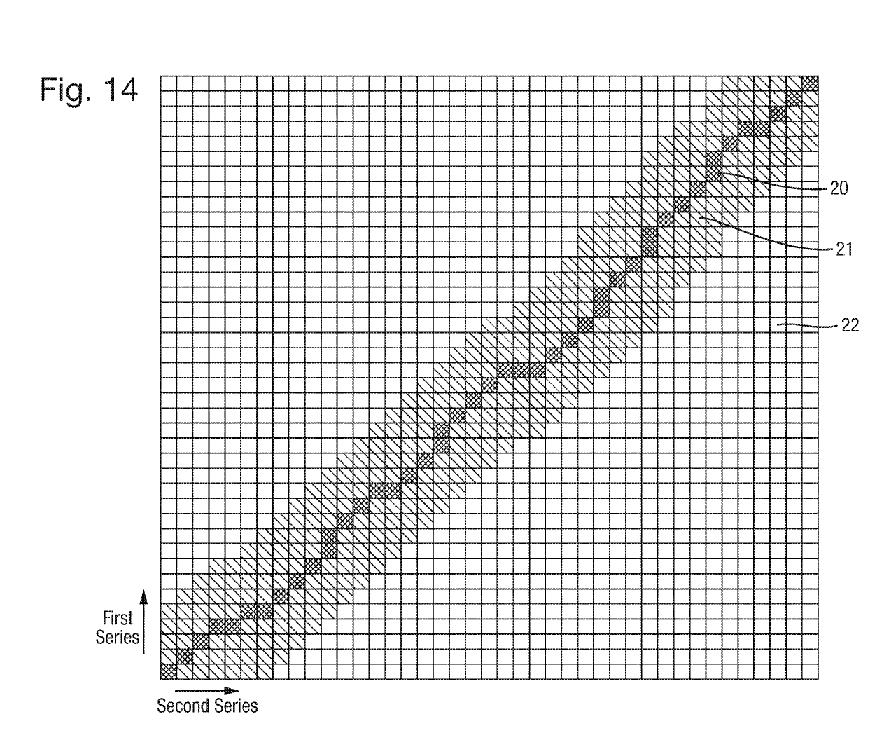

FIG. 14 is a grid of possible alignments between two series of 40 measurements;

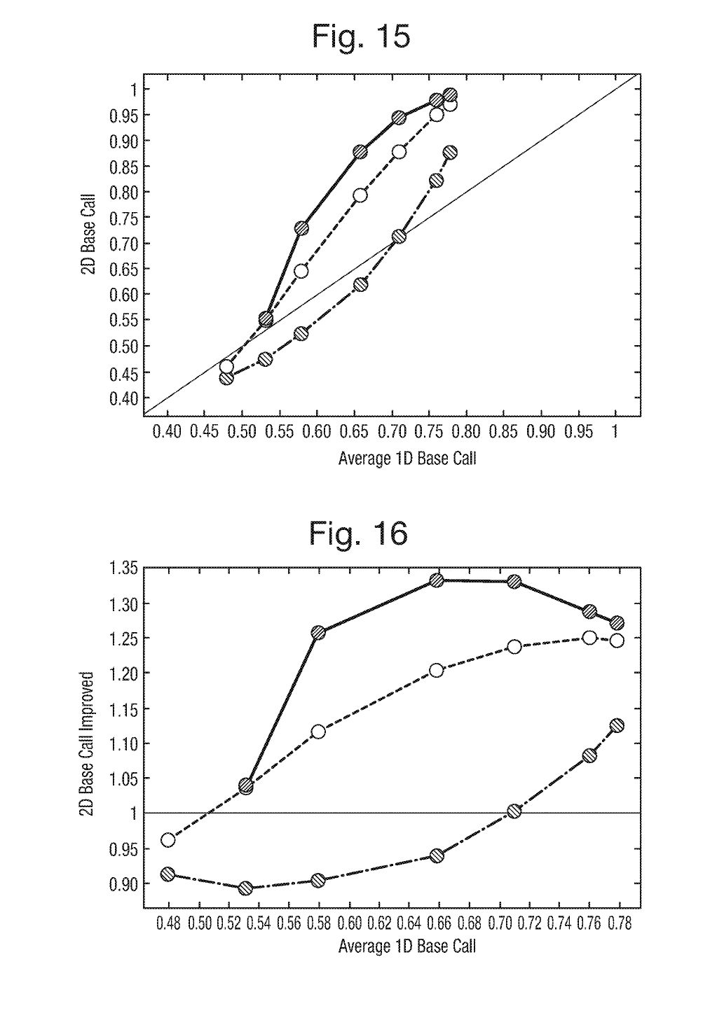

FIGS. 15 and 16 are plots of base-call quality for some simulated data;

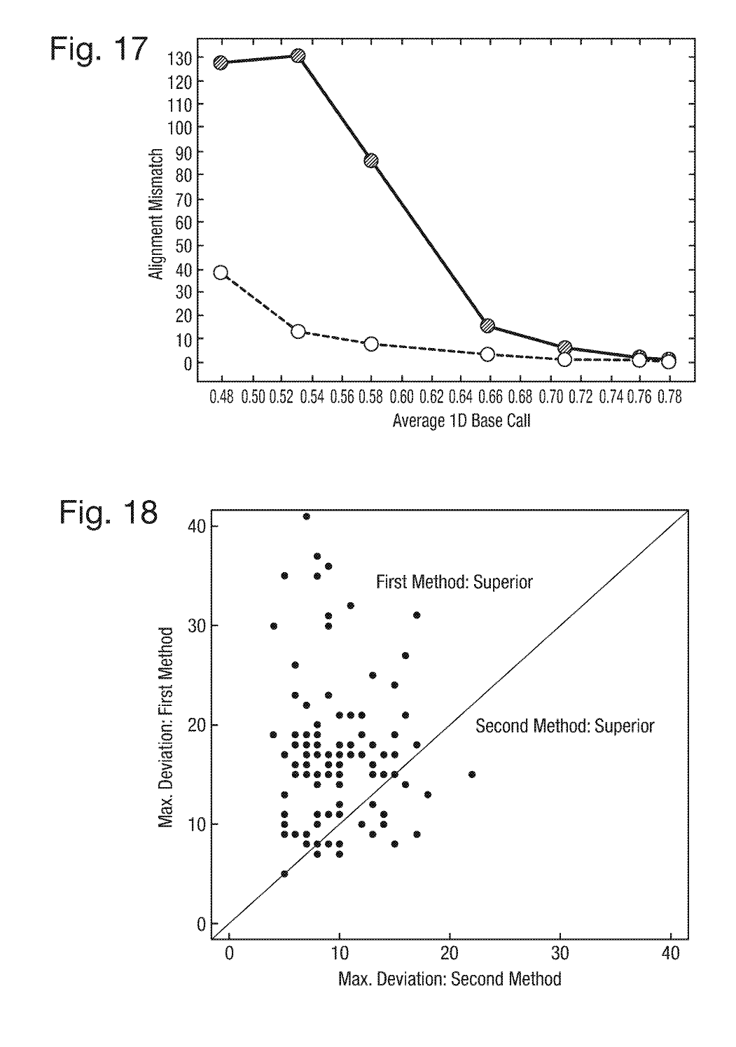

FIG. 17 is a plot of alignment mismatch for some simulated data; and

FIG. 18 is a plot of scores indicating the accuracy of two alignment mappings estimated by the first and second methods of FIGS. 10 and 11 for 100 sets of simulated measurements.

A number of nucleotide and amino acid sequences may be of use in the described embodiments. In particular:

SEQ ID NO: 1 is the nucleotide sequence encoding the pore MS-(B1)8 (=MS-(D90N/D91N/D93N/D118R/D134R/E139K)8).

SEQ ID NO: 2 is the amino acid sequence encoding the pore MS-(B1)8 (=MS-(D90N/D91N/D93N/D118R/D134R/E139K)8).

SEQ ID NO: 3 is the nucleotide sequence encoding the pore MS-(B2)8 (=MS-(L88N/D90N/D91N/D93N/D118R/D134R/E139K)8).

SEQ ID NO: 4 is the amino acid sequence encoding the pore MS-(B2)8 (=MS-(L88N/D90N/D91N/D93N/D118R/D134R/E139K)8). The amino acid sequence of B2 is identical to that of B1 except for the mutation L88N.

SEQ ID NO: 5 is the sequence for wild type E. coli Exonuclease I (WT EcoExo I), a preferred polynucleotide handling enzyme.

SEQ ID NO: 6 is the sequence for E. coli Exonuclease III, a preferred polynucleotide handling enzyme.

SEQ ID NO: 7 is the sequence for T. thermophilus RecJ, a preferred polynucleotide handling enzyme.

SEQ ID NO: 8 is the sequence for bacteriophage lambda exonuclease, a preferred polynucleotide handling enzyme.

SEQ ID NO: 9 is the sequence for Phi29 DNA polymerase, a preferred polynucleotide handling enzyme.

Aspects of the following method use techniques disclosed in WO-2013/041878, but with modifications relating to the generation and use of an estimated alignment mapping between the measurements of the respective series. Accordingly reference is made to WO-2013/041878, which is incorporated herein by reference.

FIG. 1 shows a method of generating an estimate of a target sequence of polymer units.

In step S1, plural series of raw measurements 11 are taken from respective sequences of polymer units. Step S1 is performed by a measurement system 8 configured to take the measurements. The raw measurements 11 taken from the sequences of polymer units in step S1 are supplied as input signals to an analysis unit 9 for analysis. A series of raw measurements 11 from each of the respective sequence of polymer units is supplied.

The nature of an individual sequences of polymer units from which measurements are taken is as follows. The various features described below are examples and not limitative. Also, the features described are not necessarily applied together and may be applied in any combination.

Each given polymer unit may be of different types (or identities), depending on the nature of the polymer.

The polymer may be a polynucleotide (or nucleic acid), a polypeptide such as a protein, a polysaccharide, or any other polymer. The polymer may be natural or synthetic. The polymer units may be nucleotides. The nucleotides may be of different types that include different nucleobases.

The polynucleotide may be deoxyribonucleic acid (DNA), ribonucleic acid (RNA), cDNA or a synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains. The polynucleotide may be single-stranded, be double-stranded or comprise both single-stranded and double-stranded regions. Typically cDNA, RNA, GNA, TNA or LNA are single stranded.

The methods described herein may be used to identify any nucleotide. The nucleotide can be naturally occurring or artificial. A nucleotide typically contains a nucleobase (which may be shortened herein to “base”), a sugar and at least one phosphate group. The nucleobase is typically heterocyclic. Suitable nucleobases include purines and pyrimidines and more specifically adenine, guanine, thymine, uracil and cytosine. The sugar is typically a pentose sugar. Suitable sugars include, but are not limited to, ribose and deoxyribose. The nucleotide is typically a ribonucleotide or deoxyribonucleotide. The nucleotide typically contains a monophosphate, diphosphate or triphosphate.

The nucleotide can include a damaged or epigenetic base. The nucleotide can be labelled or modified to act as a marker with a distinct signal. This technique can be used to identify the absence of a base, for example, an abasic unit or spacer in the polynucleotide.

Of particular use when considering measurements of modified or damaged DNA (or similar systems) are the methods where complementary data are considered. The additional information provided allows distinction between a larger number of underlying states.

The method may also be applied to any other type of polymer, some non-limitative examples of which are as follows.

The polymer may be a polypeptide, in which case the polymer units may be amino acids that are naturally occurring or synthetic.

The polymer may be a polysaccharide, in which case the polymer units may be monosaccharides.

Particularly where the measurement system 8 comprises a nanopore and the polymer comprises a polynucleotide, the polynucleotide may be long, for example at least 2 kB (kilo-bases), i.e. at least 2,000 nucleotides, at least 10 kB, at least 30 kB, at least 50 kB. Herein, the term 1-mer′ refers to a group of k-polymer units, where k is a positive integer, including the case that k is one, in which the k-mer is a single polymer unit. In some contexts, reference is made to k-mers where k is a plural integer, being a subset of k-mers in general excluding the case that k is one.

Each given k-mer may therefore also be of different types, corresponding to different combinations of the different types of each polymer unit of the k-mer.

The nature of the measurements is as follows.

The measurement system 8 may be any of a range of types of device.

The measurement system 8 may be a nanopore system that comprises a nanopore. In this case, the measurements may be taken during translocation of the polymer through the nanopore. The translocation of the polymer through the nanopore generates a characteristic signal in the measured property that may be observed, and may be referred to overall as an “event”.

The nanopore is a pore, typically having a size of the order of nanometres, that allows the passage of polymers therethrough. A property that depends on the polymer units translocating through the pore may be measured. The property may be associated with an interaction between the polymer and the pore. Interaction of the polymer may occur at a constricted region of the pore. The measurement system 8 measures the property, producing a measurement that is dependent on the polymer units of the polymer.

The nanopore may be a biological pore or a solid state pore.

Where the nanopore is a biological pore, it may have the following properties.

The biological pore may be a transmembrane protein pore. Transmembrane protein pores for use in the methods described herein can be derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, outer membrane porin F (OmpF), outer membrane porin G (OmpG), outer membrane phospholipase A and Neisseria autotransporter lipoprotein (NalP). α-helix bundle pores comprise a barrel or channel that is formed from α-helices. Suitable α-helix bundle pores include, but are not limited to, inner membrane proteins and α outer membrane proteins, such as WZA and ClyA toxin. The transmembrane pore may be derived from Msp or from α-hemolysin (α-HL).

A suitable transmembrane protein pore may be derived from Msp, preferably from MspA. Such a pore will be oligomeric and typically comprises 7, 8, 9 or 10 monomers derived from Msp. The pore may be a homo-oligomeric pore derived from Msp comprising identical monomers. Alternatively, the pore may be a hetero-oligomeric pore derived from Msp comprising at least one monomer that differs from the others. The pore may also comprise one or more constructs that comprise two or more covalently attached monomers derived from Msp. Suitable pores are disclosed in WO-2012/107778. The pore may be derived from MspA or a homolog or paralog thereof.

The biological pore may be a naturally occurring pore or may be a mutant pore. Typical pores are described in WO-2010/109197, Stoddart D et al., Proc Natl Acad Sci, 12; 106(19):7702-7, Stoddart D et al., Angew Chem Int Ed Engl. 2010; 49(3):556-9, Stoddart D et al., Nano Lett. 2010 Sep. 8; 10(9):3633-7, Butler T Z et al., Proc Natl Acad Sci 2008; 105(52):20647-52, and WO-2012/107778.

The biological pore may be MS-(B1)8. The nucleotide sequence encoding B1 and the amino acid sequence of B1 are Seq ID: 1 and Seq ID: 2.

The biological pore is more preferably MS-(B2)8. The amino acid sequence of B2 is identical to that of B1 except for the mutation L88N. The nucleotide sequence encoding B2 and the amino acid sequence of B2 are Seq ID: 3 and Seq ID: 4.

The biological pore may be inserted into a membrane, such as an amphiphilic layer, for example a lipid bilayer. An amphiphilic layer is a layer formed from amphiphilic molecules, such as phospholipids, which have both hydrophilic and lipophilic properties. The amphiphilic layer may be a monolayer or a bilayer. The amphiphilic layer may be a co-block polymer such as disclosed by (Gonzalez-Perez et al., Langmuir, 2009, 25, 10447-10450). Alternatively, a biological pore may be inserted into a solid state layer.

Alternatively, the nanopore may be a solid state pore comprising an aperture formed in a solid state layer. In that case, it may have the following properties.

Such a solid state layer is typically not of biological origin. In other words, a solid state layer is typically not derived from or isolated from a biological environment such as an organism or cell, or a synthetically manufactured version of a biologically available structure. Solid state layers can be formed from both organic and inorganic materials including, but not limited to, microelectronic materials, insulating materials such as Si3N4, Al203, and SiO, organic and inorganic polymers such as polyamide, plastics such as Teflon® or elastomers such as two-component addition-cure silicone rubber, and glasses. The solid state layer may be formed from graphene. Suitable graphene layers are disclosed in WO-2009/035647 and WO-2011/046706.

When the solid state pore is an aperture in a solid state layer, the aperture may be modified, chemically, or otherwise, to enhance its properties as a nanopore.

A solid state pore may be used in combination with additional components which provide an alternative or additional measurement of the polymer such as tunnelling electrodes (Ivanov A P et al., Nano Lett. 2011 Jan. 12; 11(1):279-85), or a field effect transistor (FET) device (WO-2005/124888). Suitable solid state pores may be formed by known processes including for example those described in WO-00/79257.

In one type of measurement system 8, there may be used current measurements of the ion current flowing through a nanopore. These and other electrical measurements may be made using standard single channel recording equipment as describe in Stoddart D et al., Proc Natl Acad Sci, 12; 106(19):7702-7, Lieberman K R et al, J Am Chem Soc. 2010; 132(50):17961-72, and WO-2000/28312. Alternatively, electrical measurements may be made using a multi-channel system, for example as described in WO-2009/077734 and WO-2011/067559.

In order to allow measurements to be taken as the polymer translocates through a nanopore, the rate of translocation can be controlled by a polymer binding moiety. Typically the moiety can move the polymer through the nanopore with or against an applied field. The moiety can be a molecular motor using for example, in the case where the moiety is an enzyme, enzymatic activity, or as a molecular brake. Where the polymer is a polynucleotide there are a number of methods proposed for controlling the rate of translocation including use of polynucleotide binding enzymes. Suitable enzymes for controlling the rate of translocation of polynucleotides include, but are not limited to, polymerases, helicases, exonucleases, single stranded and double stranded binding proteins, and topoisomerases, such as gyrases. For other polymer types, moieties that interact with that polymer type can be used. The polymer interacting moiety may be any disclosed in WO-2010/086603, WO-2012/107778, and Lieberman K R et al, J Am Chem Soc. 2010; 132(50):17961-72), and for voltage gated schemes (Luan B et al., Phys Rev Lett. 2010; 104(23):238103).

A polymer binding moiety can be used in a number of ways to control the polymer motion. The moiety can move the polymer through the nanopore with or against the applied field. The moiety can be used as a molecular motor using for example, in the case where the moiety is an enzyme, enzymatic activity, or as a molecular brake. The translocation of the polymer may be controlled by a molecular ratchet that controls the movement of the polymer through the pore. The molecular ratchet may be a polymer binding protein.

For polynucleotides, the polynucleotide binding protein is preferably a polynucleotide handling enzyme. A polynucleotide handling enzyme is a polypeptide that is capable of interacting with and modifying at least one property of a polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position. The polynucleotide handling enzyme does not need to display enzymatic activity as long as it is capable of binding the target polynucleotide and controlling its movement through the pore. For instance, the enzyme may be modified to remove its enzymatic activity or may be used under conditions which prevent it from acting as an enzyme. Such conditions are discussed in more detail below.

The polynucleotide handling enzyme may be derived from a nucleolytic enzyme. The polynucleotide handling enzyme used in the construct of the enzyme is more preferably derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31. The enzyme may be any of those disclosed in WO-2010/086603.

Preferred enzymes are polymerases, exonucleases, helicases and topoisomerases, such as gyrases. Suitable enzymes include, but are not limited to, exonuclease I from E. coli (Seq ID: 5), exonuclease III enzyme from E. coli (Seq ID: 6), RecJ from T. thermophilus (Seq ID: 7) and bacteriophage lambda exonuclease (Seq ID: 8) and variants thereof. Three subunits comprising the sequence shown in Seq ID: 8 or a variant thereof interact to form a trimer exonuclease. The enzyme is preferably derived from a Phi29 DNA polymerase. An enzyme derived from Phi29 polymerase comprises the sequence shown in Seq ID: 9 or a variant thereof.

A variant of Seq IDs: 5, 6, 7, 8 or 9 is an enzyme that has an amino acid sequence which varies from that of Seq IDs: 5, 6, 7, 8 or 9 and which retains polynucleotide binding ability. The variant may include modifications that facilitate binding of the polynucleotide and/or facilitate its activity at high salt concentrations and/or room temperature.

Over the entire length of the amino acid sequence of Seq IDs: 5, 6, 7, 8 or 9, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of Seq IDs: 5, 6, 7, 8 or 9 over the entire sequence. There may be at least 80%, for example at least 85%, 90% or 95%, amino acid identity over a stretch of 200 or more, for example 230, 250, 270 or 280 or more, contiguous amino acids (“hard homology”). Homology is determined as described above. The variant may differ from the wild-type sequence in any of the ways discussed above with reference to Seq ID: 2. The enzyme may be covalently attached to the pore as discussed above.

Suitable strategies for single strand DNA sequencing are the translocation of the DNA through a nanopore, both cis to trans and trans to cis, either with or against an applied potential. The most advantageous mechanism for strand sequencing is the controlled translocation of single strand DNA through the nanopore under an applied potential. Exonucleases that act progressively or processively on double stranded DNA can be used on the cis side of the pore to feed the remaining single strand through under an applied potential or the trans side under a reverse potential. Likewise, a helicase that unwinds the double stranded DNA can also be used in a similar manner. There are also possibilities for sequencing applications that require strand translocation against an applied potential, but the DNA must be first “caught” by the enzyme under a reverse or no potential. With the potential then switched back following binding the strand will pass cis to trans through the pore and be held in an extended conformation by the current flow. The single strand DNA exonucleases or single strand DNA dependent polymerases can act as molecular motors to pull the recently translocated single strand back through the pore in a controlled stepwise manner, trans to cis, against the applied potential. Alternatively, the single strand DNA dependent polymerases can act as molecular brake slowing down the movement of a polynucleotide through the pore. Any moieties, techniques or enzymes described in WO-2012/107778 or WO-2012/033524 could be used to control polymer motion.

In general, when the measurement is current measurement of ion current flow through a nanopore, the ion current may typically be the DC ion current, although in principle an alternative is to use the AC current flow (i.e. the magnitude of the AC current flowing under application of an AC voltage).

However, alternative types of the measurement system 8 and measurements are also possible. Some non-limitative examples of alternative types of the measurement system 8 are as follows.

The measurement system 8 may be a scanning probe microscope. The scanning probe microscope may be an atomic force microscope (AFM), a scanning tunnelling microscope (STM) or another form of scanning microscope.

In the case where the reader is an AFM, the resolution of the AFM tip may be less fine than the dimensions of an individual polymer unit. As such the measurement may be a function of multiple polymer units. The AFM tip may be functionalised to interact with the polymer units in an alternative manner to if it were not functionalised. The AFM may be operated in contact mode, non-contact mode, tapping mode or any other mode.

In the case where the reader is a STM the resolution of the measurement may be less fine than the dimensions of an individual polymer unit such that the measurement is a function of multiple polymer units. The STM may be operated conventionally or to make a spectroscopic measurement (STS) or in any other mode.

The measurement system 8 may take optical measurements. A suitable optical method involving the measurement of fluorescence is disclosed by J. Am. Chem. Soc. 2009, 131 1652-1653.

The measurement system 8 may take electrical measurements of types other than current measurements of ion current through a nanopore as described above. Possible electrical measurement include: current measurements, impedance measurements, tunnelling measurements (for example as disclosed in Ivanov A P et al., Nano Lett. 2011 Jan. 12; 11(1):279-85), and field effect transistor (FET) measurements (for example as disclosed in WO2005/124888).

Optical measurements may be combined with electrical measurements (Soni G V et al., Rev Sci Instrum. 2010 January; 81(1):014301).

The measurement system 8 may take measurements of different natures. The measurement may be of different natures because they are measurements of different physical properties, which may be any of those described above. Alternatively, the measurements may be of different natures because they are measurements of the same physical properties but under different conditions, for example electrical measurements such as current measurements under different bias voltages.

The plural series of measurements may be of different types made concurrently on the same region of the same polymer, for example: electrical measurements made under different conditions; a trans-membrane current measurement and a FET measurement made at the same time; or an optical measurement and an electrical measurement made at the same time (Heron A J et al., J Am Chem Soc. 2009; 131(5):1652-3). In that case, the resultant series of raw measurements 11 may comprise a series of a predetermined plural number of measurements of different natures that are dependent on the same k-mer.

Each measurement is dependent on a k-mer, being k polymer units of the respective sequence of polymer units, where k is a positive integer. Although ideally the measurements would be dependent on a single polymer unit (i.e. where k is one), with many typical types of the measurement system 8, each measurement is dependent on a k-mer of plural polymer units (i.e. where k is a plural integer). That is, each measurement is dependent on the sequence of each of the polymer units in the k-mer where k is a plural integer.

Typically the measurements are of a property that is associated with an interaction between the polymer and the measurement system 8. The advantages described herein are particularly achieved when applied to measurements that are dependent on k-mers where k is a plural integer. The analysis method is described below for the case that the measurements are dependent on a k-mer where k is two or more, but the same method may be applied in simplified form to measurements that are dependent on a k-mer where k is one.

In some cases, it is preferred to use measurements that are dependent on small groups of polymer units, for example doublets or triplets of polymer units (i.e. in which k=2 or k=3). In other cases, it is preferred to use measurements that are dependent on larger groups of polymer units, i.e. with a “broad” resolution. Such broad resolution may be particularly useful for examining homopolymer regions.

Especially where measurements are dependent on a k-mer where k is a plural integer, it is desirable that the measurements are resolvable (i.e. separated) for as many as possible of the possible k-mers. Typically this can be achieved if the measurements produced by different k-mers are well spread over the measurement range and/or have a narrow distribution. This may be achieved to varying extents by different types of the measurement system 8. However, it is a particular advantage that it is not essential for the measurements produced by different k-mers to be resolvable.

FIG. 2 schematically illustrates an example of a measurement system 8 comprising a nanopore that is a biological pore 1 inserted in a biological membrane 2 such as an amphiphilic layer. A polymer 3 comprising a series of polymer units 4 is translocated through the biological pore 1 as shown by the arrows. In this example, the polymer 3 may be a polynucleotide in which the polymer units 4 are nucleotides, as described above. The polymer 3 interacts with an active part 5 of the biological pore 1 causing an electrical property such as the trans-membrane current to vary in dependence on a k-mer inside the biological pore 1. In this example, the active part 5 is illustrated as interacting with a k-mer of three polymer units 4, but this is not limitative.

Electrodes 6 arranged on each side of the biological membrane 2 are connected to a an electrical circuit 7, including a control circuit 71 and a measurement circuit 72.

The control circuit 71 is arranged to supply a voltage to the electrodes 6 for application across the biological pore 1.

The measurement circuit 72 is arranged to measures the electrical property. Thus the measurements are dependent on the k-mer inside the biological pore 1.

The plural series of measurements may each be made by the same technique or may be made by different techniques. The plural series of measurements may be made using the same or different types of the measurement system 8. Multiple measurements can be made one after the other by translocating a given polymer or regions thereof through the pore more than once. These measurements can be the same measurement or different measurements and conducted under the same conditions, or under different conditions.

A typical form of the signal output by many types of the measurement system 8 as the series of raw measurements 11 to be analysed is a “noisy step wave”, although without limitation to this signal type. An example of a series of raw measurements 11 having this form is shown in FIG. 3 for the case of an ion current measurement obtained using a type of the measurement system 8 comprising a nanopore.

This type comprises an input series of measurements in which successive groups of plural measurements are dependent on the same k-mer. The plural measurements in each group are of a constant value, subject to some variance discussed below, and therefore form a “level” in the series of raw measurements 11. Such a level may typically be formed by the measurements being dependent on the same k-mer (or successive k-mers of the same type) and hence correspond to a common state of the measurement system 8.

The signal moves between a set of levels, which may be a large set. Given the sampling rate of the instrumentation and the noise on the signal, the transitions between levels can be considered instantaneous, thus the signal can be approximated by an idealised step trace.

The measurements corresponding to each state are constant over the time scale of the event, but for most types of the measurement system 8 will be subject to variance over a short time scale. Variance can result from measurement noise, for example arising from the electrical circuits and signal processing, notably from the amplifier in the particular case of electrophysiology. Such measurement noise is inevitable due the small magnitude of the properties being measured. Variance can also result from inherent variation or spread in the underlying physical or biological system of the measurement system 8. Most types of the measurement system 8 will experience such inherent variation to greater or lesser extents. For any given types of the measurement system 8, both sources of variation may contribute or one of these noise sources may be dominant.

In addition, typically there is no a priori knowledge of number of measurements in the group, this varying unpredictably.

These two factors of variance and lack of knowledge of the number of measurements can make it hard to distinguish some of the groups, for example where the group is short and/or the levels of the measurements of two successive groups are close to one another.

The series of raw measurements 11 may take this form as a result of the physical or biological processes occurring in the measurement system 8. Thus, in some contexts each group of measurements may be referred to as a “state”.

For example, in some types of the measurement system 8 comprising a nanopore, the event consisting of translocation of the polymer through the nanopore may occur in a ratcheted manner. During each step of the ratcheted movement, the ion current flowing through the nanopore at a given voltage across the nanopore is constant, subject to the variance discussed above. Thus, each group of measurements is associated with a step of the ratcheted movement. Each step corresponds to a state in which the polymer is in a respective position relative to the nanopore. Although there may be some variation in the precise position during the period of a state, there are large scale movements of the polymer between states. Depending on the nature of the measurement system 8, the states may occur as a result of a binding event in the nanopore.

The duration of individual states may be dependent upon a number of factors, such as the potential applied across the pore, the type of enzyme used to ratchet the polymer, whether the polymer is being pushed or pulled through the pore by the enzyme, pH, salt concentration and the type of nucleoside triphosphate present. The duration of a state may vary typically between 0.5 ms and 3 s, depending on the measurement system 8, and for any given nanopore system, having some random variation between states. The expected distribution of durations may be determined experimentally for any given measurement system 8.

As discussed further below, the method uses plural input series of measurements each of which may take the form described above in which successive groups of plural measurements in each series are dependent on the same k-mer. Although each of the series of measurements correspond to the target sequence, the present methods apply to plural series that are not registered so that it is not known a priori which measurements from the respective series correspond and are dependent on the same k-mer. This might be the case, for example, if the series of measurements are taken by different measurement systems 8 and/or at different times.

The extent to which a given measurement system 8 provides measurements that are dependent on k-mers and the size of the k-mers may be examined experimentally. Possible approaches to this are disclosed in WO-2013/041878.

The respective sequences of polymer units from which different series of measurements are taken will now be discussed.

Each of the respective sequences of polymer units correspond to the target sequence of polymer units which is to be estimated.

Any or all of the respective sequences of polymer units may correspond to the target sequence by actually comprising the target sequence.

Similarly, any or all of them may correspond to the target sequence by having a predetermined relationship with the target sequence. The respective sequences of polymer units may include sequences that comprise the target sequence and/or sequences that have a predetermined relationship with the target sequence, in any combination.

The respective sequences of polymer units from which measurements are taken may be in the same or different polymers, that is physically the same or different polymer.

In the case of the respective sequences of polymer units being in the same polymer, they may be the same sequence measured repeatedly under the same or different conditions.

In the case of the respective sequences of polymer units being in the same polymer, they may be different parts of the polymer, typically measured sequentially. In the latter case, the sequences may each be the same sequence, typically the target sequence, or may be the target sequence and one or more sequences that are related to the target sequence.

In the case of the respective sequences of polymer units being in different polymers, they may be polymers in the same sample measured in a common operation of the measurement system 8 or may be in different samples that are measured by the same or different measurement systems 8. For example, in the case that the measurement system 8 uses a nanopore, the measurements may be measurements of the same sequence using different nanopores, for example that provide with different measurement-sequence characteristics.

In the case of the respective sequences of polymer units being in different polymers, they may be polymers that are prepared by a process causing each to include the target sequence or by a process causing different polymers to include the target sequence and one or more sequences that are related to the target sequence.

In the case of the respective sequences including sequences having a predetermined relationship with the target sequence, that relationship may be that the sequences are complementary to the target sequence. This may be referred to as “template-complement”, template referring to the target sequence and the complementary sequence. For example nucleobase A pairs with nucleobase T and nucleobase C pairs with nucleobase G. Thus a template polynucleotide with a sequence AACTC would have a complementary sequence of TTGAG.

As an example of using a complementary sequence, there may be used techniques proposed for polynucleotides such as DNA, in which the template and the complement sequences are provided in the same polymer, linked by a bridging moiety such as a hairpin loop. When the template and complement sequences are attached by a hairpin, the complement sequence of nucleotides is measured in reverse order in the nanopore to that of the template, but this is straightforward to handle in the methods described below, simply by reversing the order of one of the series of measurements.

In that case, the template and complement regions may be hybridized in the sample, and may be dehybridized prior to translocation through a nanopore in which measurements are taken. Such dehybridization may be performed using a polynucleotide binding protein, for example a helicase.

Measurements may be taken as disclosed in WO2013/014451.

Methods for forming template-complement nucleotide sequences may also be carried out as disclosed in WO-2010/086622.

When present, the hairpin may comprise an identifier to distinguish between the template and complement strands. The identifier will typically provide a readily identifiable and unique signal that may be distinguished from the template and complement regions. The identifier may comprise for example a known sequence of natural or non-natural polynucleotides, one or more abasic residues or one or more modified bases. The identifier may comprise one or more spacers which are capable of stalling a DNA processing enzyme such as a helicase, wherein the DNA processing enzyme is able to move past the one or more spacers following application of a potential difference across a nanopore and moving the template and complement strands through the nanopore. The one or more spacers may comprise peptide nucleic acid (PNA), glycerol nucleic acid (GNA), threose nucleic acid (TNA), locked nucleic acid (LNA) or a synthetic polymer with nucleotide side chains.

Despite this plural dimensional example for the case of template-complement polynucleotides such as DNA, other relationships between the sequences may be used in a plural dimensional approach. An example of another type of relationship is structural information in polymers. This information may exist in RNA, which is known to form functional structures. This information may also exist in polypeptides (proteins). In the case of proteins the structural information may be related to hydrophobic or hydrophilic regions. The information may also be about alpha helical, beta sheet or other secondary structures. The information may be about known functional motifs such as binding sites, catalytic sites and other motifs.

Another example of a relationship is that one of the sequences is DNA and the other sequence is cDNA.

The analysis of the series of raw measurements 11 by the analysis unit 9 will now be described. The analysis unit 9 forms an analysis system, either by itself or with other units.

The analysis is performed in steps S2 to S4 that are implemented in the analysis unit 9 illustrated schematically in FIG. 1. The analysis unit 9 receives and analyses the series of raw measurements 11 taken by, and supplied from, the measurement system 8. The analysis unit 9 and the measurement system 8 together constitute an apparatus for analysing a polymer. The analysis unit 9 may also provide control signals to the control circuit 7, for example to select the voltage applied across the biological pore 1 in the measurement system 8. The series of raw measurements 11 may be supplied over any suitable connection, for example a direct connection in the case that the analysis unit 9 and the measurement system 8 are physically located together, or any type of network connection in the case that the analysis unit 9 and the measurement system 8 are physically remote from each other.

The apparatus including the analysis unit 9 and the measurement system 8 may be arranged as disclosed in any of WO-2008/102210, WO-2009/07734, WO-2010/122293 and/or WO-2011/067559.

The analysis unit 9 may be implemented by a computer apparatus executing a computer program or may be implemented by a dedicated hardware device, or any combination thereof. In either case, the data used by the method may be stored in a memory 10 in the analysis unit 9. The computer apparatus, where used, may be any type of computer system but is typically of conventional construction. The computer program may be written in any suitable programming language. The computer program may be stored on a computer-readable storage medium, which may be of any type, for example: a recording medium which is insertable into a drive of the computing system and which may store information magnetically, optically or opto-magnetically; a fixed recording medium of the computer system such as a hard drive; or a computer memory.

The method is performed on the series of raw measurements 11 that each comprises a series of measurements of the type described above comprising successive groups of plural measurements that are dependent on the same k-mer without a priori knowledge of number of measurements in any group.

In a state detection step S2, each series of raw measurements 11 is processed to identify successive groups of raw measurements and to derive a series of measurements 12 consisting of a predetermined number of measurements in respect of each identified group. Thus, a series of measurements 12 is derived in respect of each sequence of polymer units that is measured. Further analysis is performed in steps S3 to S5 on the thus derived series of measurements 12.

The purpose of the state detection step S2 is to reduce the series of raw measurements to a predetermined number of measurements associated with each k-mer to simplify the subsequent analysis. For example a noisy step wave signal, as shown in FIG. 3 may be reduced to states where a single measurement associated with each state may be the mean current. This state may be termed a level.

The state detection step S2 may be performed on each individual series of raw measurements 11 using the method shown in FIG. 4 that looks for short-term increases in the derivative of the series of raw measurements 11 as follows.

In step S2-1, the series of raw measurements 11 is differentiated to derive its derivative.

In step S2-2, the derivative from step S2-1 is subjected to low-pass filtering to suppress high-frequency noise, which the differentiation in step S2-1 tends to amplify.

In step S2-3, the filtered derivative from step S2-2 is thresholded to detect transition points between the groups of measurements, and thereby identify the groups of raw measurements.

In step S2-4, a predetermined number of measurements is derived from each group of raw measurements identified in step S2-3. The measurements output from step S2-4 form the series of measurements 12.

The predetermined number of measurements may be one or more.

In the simplest approach, a single measurement is derived from each group of raw measurements, for example the mean, median, standard deviation or number, of raw measurements in each identified group.

In other approaches, a predetermined plural number of measurements of different natures are derived from each group, for example any two or more of the mean, median, standard deviation or number of raw measurements in each identified group. In that case, the a predetermined plural number of measurements of different natures are taken to be dependent on the same k-mer since they are different measures of the same group of raw measurements.

The state detection step S2 may use different methods from that shown in FIG. 4. For example a common simplification of method shown in FIG. 4 is to use a sliding window analysis which compares the means of two adjacent windows of data. A threshold can then be either put directly on the difference in mean, or can be set based on the variance of the data points in the two windows (for example, by calculating Student's t-statistic). A particular advantage of these methods is that they can be applied without imposing many assumptions on the data.

Other information associated with the measured levels can be stored for use later in the analysis. Such information may include without limitation any of: the variance of the signal; asymmetry information; the confidence of the observation; the length of the group.

By way of example, FIG. 5 illustrates an experimentally determined series of raw measurements 11 reduced by a moving window t-test. In particular, FIG. 5 shows the series of raw measurements 11 as the light line. Levels following state detection are shown overlayed as the dark line. FIG. 6 shows the series of measurements 12 derived for the entire trace, calculating the level of each state from the mean value between transitions.

However, as described in more detail below, the state detection step S2 is optional and may be omitted in an alternative described further below. In this case, the further analysis is performed on the respective series of raw measurements 11 themselves, instead of the series of measurements 12. Although, the following description refers to method performed on the series of measurements 12, in this alternative that the state detection step S2 is omitted, then the references to the steps performed on the series of measurements 12 are instead performed on the series of raw measurements 11 themselves, instead of the series of measurements 12. Similarly, in this case the series of raw measurements 11 form the “series of measurements” recited in the definition of the invention.

The method uses a model 13 in respect of each series of measurements 12, the model 13 being stored in the memory 10 of the analysis unit 9. The model of each series of measurements 11 that treat the measurements as observations of a series of k-mer states of different possible types. The k-mer states of the model 13 may model the actual k-mers on which the measurements depend, although mathematically this is not necessary and so the k-mer states may be an abstraction of the actual k-mers. Thus, the different types of k-mer states may correspond to the different types of k-mers that exist in the sequence of polymer units. Alternatively, there may be a larger number of k-mer states than types of k-mer. For example, this may be the case when it is desired to model different physical conditions experienced by the same k-mer at the time a measurement is taken.

The mathematical basis of the model 13 of a single series of measurements will now be considered.

The relationship between a sequence of random variables {T₁, T₂, . . . , Tₙ} from which currents are sampled may be represented by a simple model A, which represents the conditional independence relationships between variables T₁ to Tₙ.

Each current measurement is dependent on a k-mer being read, so there is an underlying set of random variables {S₁, S₂, . . . , Sₙ} representing the underlying sequence of k-mers with a corresponding model B which relates each random variable S₁ to Sₙ to the corresponding one of the variables T₁ to Tₙ.

These models as applied to the current area of application may take advantage of the Markov property. In model A, if f(Tᵢ) is taken to represent the probability density function of the random variable Tᵢ, then the Markov property can be represented as:

f(Tₘ|Tₘ₋₁)=f(Tₘ|T₁,T₂, . . . ,Tₘ₋₁)

In model B, the Markov property can be represented as:

P(Sₘ|Sₘ₋₁)=P(Sₘ|S₁,S₂, . . . ,Sₘ₋₁)

Depending on exactly how the problem is encoded, natural methods for solution may include Bayesian networks, Markov random fields, Hidden Markov Models, and also including variants of these models, for example conditional or maximum entropy formulations of such models. Methods of solution within these slightly different frameworks are often similar.

Generally, the model 13 comprises transition weightings 14 and emission weightings 15 in respect of a series of k-mer states corresponding to the series of measurements 12.

The transition weightings 14 are provided in respect of each transition between successive k-mer states in the series of k-mer states. Each transition may be considered to be from an origin k-mer state to a destination k-mer state. The transition weightings 14 represent the relative weightings of possible transitions between the possible types of the k-mer state, that is from an origin k-mer state of any type to a destination k-mer state of any type. In general, this includes a weighting for a transition between two k-mer states of the same type.

The emission weightings 15 are provided in respect of each type of k-mer state. The emission weightings 15 are weightings for different measurements being observed when the k-mer state is of that type. Conceptually, the emission weightings 15 may be thought of as representing the chances of the chances of observing given values of measurements for that k-mer state, although they do not need to be probabilities.

Conceptually, the transition weightings 14 may be thought of as representing the chances of the possible transition, although they do not need to be probabilities. Therefore, the transition weightings 14 take account of the chance of the k-mer state on which the measurements depend transitioning between different k-mer states, which may be more and less likely depending on the types of the origin and destination k-mer states.

In particular, the measurements from individual k-mers are not required to be resolvable from each other, and it is not required that there is a transform from groups of k measurements that are dependent on the same polymer unit to a value in respect of that transform, i.e. the set of observed states is not required to be a function of a smaller number of parameters (although this is not excluded). Instead, the use of the model 13 provides accurate estimation by taking plural measurements into account in the consideration of the likelihood predicted by the model 13 of the series of measurements being produced by sequences of polymer units. Conceptually, the transition weightings 14 may be viewed as allowing the model 13 to take account, in the estimation of any given polymer unit, of at least the k measurements that are dependent in part on that polymer unit, and indeed also on measurements from greater distances in the sequence. The model 13 may effectively take into account large numbers of measurements in the estimation of any given polymer unit, giving a result that may be more accurate.

Similarly, the use of such a model 13 may allow the analytical technique to take account of missing measurements from a given k-mer and/or to take account of outliers in the measurement produced by a given k-mer. This may be accounted for in the transition weightings 14 and/or emission weightings 15. For example, the transition weightings 14 may represent non-zero chances of at least some of the non-preferred transitions and/or the emission weightings may represent non-zero chances of observing all possible measurements.

By way of example and without limitation, the model may be a Hidden Markov Model in which the transition weightings 14 and emission weightings 15 are probabilities. An explanation will now be given in this example.

The Hidden Markov Model (HMM) is a natural representation in the setting given here. In a HMM, the relationship between the discrete random variables Sₘ and Sₘ₊₁, where m represents the m-th k-mer state, is defined in terms of a transition matrix of transition weightings 14 that in this case are probabilities representing the probabilities of possible transitions between the possible types of k-mer state that each random variable can take, that is from origin k-mer states to destination k-mer states. For example, conventionally the (i,j)th entry of the transition matrix, where i and j represent the types of k-mer state, is a transition weighting 14 representing the probability that Sₘ₊₁=s_{m+1,j}, given that Sₘ=s_{m,i}. i.e. the probability of transitioning to the j-th possible value of Sₘ₊₁ given that Sₘ takes on its i-th possible value.

FIG. 7 is a pictorial representation of the transition matrix in respect of one transition between successive k-mer states, Sₘ to Sₘ₊₁. Here Sₘ and Sₘ₊₁ only show 4 values (types of k-mer state) for sake of illustration, but in reality there would be as many values as there are different k-mer states. Each edge represents a possible transition between the 4 possible types of k-mer states, i.e. 16 possible transitions in total. The possible transitions may be labelled with the entry from the transition matrix representing the transition probability. In FIG. 7, the probabilities of the four transitions connecting each node (type of k-mer state) in the Sₘ layer to a node (type of k-mer state) in the Sₘ₊₁ layer would classically sum to one, although non-probabilistic weightings may alternatively be used.

In general, it is desirable that the transition weightings 14 comprise values of non-binary variables (non-binary values). This allows the model 13 to represent the actual probabilities of transitions between the k-mer states.

Considering that the model 13 represents the k-mer states, any given k-mer state has k preferred transitions, being transitions from origin k-mer states to destination k-mer states that have a sequence in which the first (k−1) polymer units of the destination k-mer state are the final (k−1) polymer units of the origin k-mer state. For example in the case of polynucleotides consisting of the 4 nucleotides G, T, A and C, the origin 3-mer TAC has preferred transitions to the 3-mers ACA, ACC, ACT and ACG. To a first approximation, conceptually one might consider that the transition probabilities of the four preferred transitions are equal being (0.25) and that the transition probabilities of the other non-preferred transitions are zero, the non-preferred transitions being transitions from origin k-mer states to destination k-mer states that have a sequence different from the origin k-mer state and in which the first (k−1) polymer units are not the final (k−1) polymer units of the origin k-mer state. However, whilst this approximation is useful for understanding, the actual chances of transitions may in general vary from this approximation in any given measurement system 8. This can be reflected by the transition weightings 14 taking values of non-binary variables (non-binary values). Some examples of such variation that may be represented are as follows.

One example is that the transition probabilities of the preferred transitions might not be equal. This allows the model 13 to represent polymers in which there is an interrelationship between polymers in a sequence.

Another example is that the transition probabilities of at least some of the non-preferred transitions might be non-zero. This allows the model 13 to take account of missed measurements, that is in which there is no measurement that is dependent on one (or more) of the k-mers in the actual polymer. Such missed measurements might occur either due to a problem in the measurement system 8 such that the measurement is not physically taken, or due to a problem in the subsequent data analysis, such as the state detection step S2 failing to identify one of the groups of measurements, for example because a given group is too short or two groups do not have sufficiently separated levels.

Notwithstanding the generality of allowing the transition weightings 14 to have any value, typically it will be the case that the transition weightings 14 represent non-zero chances of the preferred transitions from origin k-mer states to destination k-mer states that have a sequence in which the first (k−1) polymer units of the destination k-mer state are the final (k−1) polymer units of the origin k-mer state, and represent lower chances of the other, non-preferred transitions. Typically also, the transition weightings 14 represent non-zero chances of at least some of said non-preferred transitions, even though the chances may be close to zero, or may be zero for some of the transitions that are absolutely excluded.

To allow for single missed k-mers in the sequence, the transition weightings 14 may represent non-zero chances of non-preferred transitions from origin k-mer states to destination k-mer states that have a sequence wherein the first (k−2) polymer units of the destination k-mer state are the final (k−2) polymer units of the origin k-mer state. For example in the case of polynucleotides consisting of 4 nucleotides, for the origin 3-mer TAC these are the transitions to all possible 3-mers starting with C. Transitions corresponding to these single missed k-mer state may be referred to as “skips”, because an intermediate k-mer state (i.e. a state starting with AC in the above example) has been skipped.

In the case of analysing the series of measurements 12 comprising a single measurement in respect of each k-mer state, then the transition weightings 14 will represent a high chance of transition for each measurement 12. Depending on the nature of the measurements, the chance of transition from an origin k-mer state to a destination k-mer state that is the same as the origin k-mer state may be zero or close to zero, or may be similar to the chance of the non-preferred transitions.

Similarly in the case of analysing a series of measurements 12 comprising a predetermined plural number of measurements in respect of each k-mer state (i.e. dependent on the same k-mer), then the transition weightings 14 may represent a low or zero chance of transition between the measurements 12 in respect of the same k-mer state. It is possible to change the transition weightings 14 to allow the origin k-mer state and destination k-mer state to be the same k-mer state. This allows, for example, for falsely detected state transitions. We may define the transitions corresponding to these repeated same k-mer states as “stays” because the k-mer state stays in the same type. In the case where all of the polymer units in the k-mer are identical, a homopolymer, a preferred transition would be a stay. In these cases the polymer has moved one position but the k-mer state remains the same.

Similarly, in the case that in the case of analysing a series of measurements 12 in which there are typically plural measurements in respect of each k-mer but of unknown quantity (which may be referred to as “sticking”), the transition weightings 14 may represent a relatively high probability of the origin k-mer state and destination k-mer state being the same k-mer state, and depending on the physical system may in some cases be larger than the probability of preferred transitions as described above being transitions from origin k-mer states to destination k-mer states in which the first (k−1) polymer units are the same as the final (k−1) polymer units of the origin k-mer state

In the an alternative that the state detection step S2 is omitted and the further analysis is performed on the respective series of raw measurements 11 themselves, instead of the series of measurements 12, then the transition weightings 14 are similar but are adapted in respect of each possible transition to represent a relatively high probability of the origin k-mer state and destination k-mer state to be the same type of k-mer state. This allows fundamentally the same use of the model 13, the adaptation of the model 13 implicitly taking account of state detection.

The emission weightings 15 in respect of each type of k-mer state will now be considered. The emission weightings 15 are for different measurements being observed when the k-mer state is of that type. In the current example of a Hidden Markov Model, the emission weightings 15 represent the probability of observing different values of measurements for that type of k-mer state. Thus, for the k-mer state m of type i represented by the node S_{m,i} in FIG. 7, the emission weighting 15 may be represented as a probability density function g(Xₘ|s_{m,i}) which describes the distribution from which measurements are observed or sampled. It is desirable that the emission weightings 15 comprise values of non-binary variables. This allows the model 13 to represent the probabilities of different measurements, that in general do not have a simple binary form.

In general, the emission weightings 15 for any given k-mer state may take any form that reflects the probability of measurements. By way of non-limitative example, the emission weightings could have distributions for the simulated coefficients that are Gaussian, triangular or square distributions, although any arbitrary distribution (including non-parametric distributions) can be defined. Different k-mer states may have emission weightings 15 with the same emission distributional form or parameterisation within a single model 13, but this is not required and different k-mer states may have different emission distributional form or parameterisation.

For many types of the measurement system 8, the measurement of a k-mer has a particular expected value that can be spread either by a spread in the physical or biological property being measured and/or by a measurement error. This can be modelled in the model 13 by using emission weightings 15 that have a suitable distribution, for example one that is unimodal.

However, for some types of the measurement system 8, the emission weightings 15 for any given type of k-mer state may be multimodal, for example arising physically from two different types of binding in the measurement system 8 and/or from the k-mer adopting multiple conformations within the measurement system 8.

Advantageously, the emission weightings 15 may represent non-zero chances of observing all possible measurements. This allows the model 13 to take account of unexpected measurements produced by a given k-mer, that are outliers. For example the emission weightings 15 probability density function may be chosen over a wide support that allows outliers with non-zero probability. For example in the case of a unimodal distribution, the emission weightings 15 for each type of k-mer state may have a Gaussian or Laplace distribution which have non-zero weighting for all real numbers.

It may be advantageous to allow the emission weightings 15 to be distributions that are arbitrarily defined, to enable elegant handling of outlier measurements and dealing with the case of a single state having multi-valued emissions.

It may be desirable to determine the emission weightings 15 empirically, for example during a training phase.

The distributions of the emission weightings 15 can be represented with any suitable number of bins across the measurement space. For example, in a case described below the distributions are defined by 500 bins over the data range. Outlier measurements can be handled by having a non-zero probability in all bins (although low in the outlying bins) and a similar probability if the data does not fall within one of the defined bins. A sufficient number of bins can be defined to approximate the desired distribution.

Thus particular advantages may be derived from the use of transition weightings 14 that represent non-zero chances of at least some of said non-preferred transitions and/or the use of emission weightings 15 that represent non-zero chances of observing all possible measurements.

Particular advantages may also be derived from the use of emission weightings 15 that correspond to the relative chance of observing a range of measurements for a given type of k-mer state. To emphasise these advantages, a simple non-probabilistic method for deriving sequence is considered as a comparative example. In this comparative example, types of k-mer state producing measurements outside a given range of the observed value are disallowed and transitions corresponding to missed measurements (skips) are disallowed, for example reducing the number of transitions in FIG. 7 by deleting edges and nodes. In the comparative example, a search is then made for the unique connected sequence of k-mer states, containing exactly one node for each Sᵢ, and corresponding to an underlying sequence of polymer units. However, as this comparative example relies on arbitrary thresholds to identify disallowed nodes and edges, it fails to find any path in the case of a skipped measurement since the appropriate edge does not exist in the graph. Similarly in the case of an outlying measurement, the comparative example will result in the corresponding node being deleted in FIG. 7, and again the correct path through the graph becomes impossible to ascertain.

In contrast a particular advantage of the use of a model 13 and an analytical technique in the alignment step S3 described below, such as a probabilistic or weighted method, is that this breakdown case can be avoided. Another advantage is that in the case where multiple allowed paths exist, the most likely, or set of likely paths can be determined.

Another particular advantage of this method relates to detection of homopolymers, that is a sequence of identical polymer units. The model-based analysis enables handling of homopolymer regions up to a length similar to the number of polymer units that contribute to the signal. For example a 6-mer measurement could identify homopolymer regions up to 6 polymer units in length.

A specific example of use of a model that is a HMM used to model and analyse data from a blunt reader head system is disclosed in WO-2013/041878.

Typically, the emission weightings 15 and transition weightings 14 are fixed at constant values, but this is not essential. As an alternative, the emission weightings 15 and/or transition weightings 14 may be varied for different sections of the measurement series to be analysed, perhaps guided by additional information about the process. As an example, an element of the matrix of transition weightings 14 which has an interpretation as a “stay” could be adjusted depending on the confidence that a particular event ( ) reflects an actual transition of the polymer. As a further example, the emission weightings 15 could be adjusted to reflect systematic drift in the background noise of the measuring device or changes made to the applied voltage. The scope of adjustments to the weightings is not limited to these examples.

Typically, there is a single representation of each k-mer (i.e. a single type of k-mer state for each type of k-mer), but this is not essential. As an alternative, the model 13 may have plural distinct representations of some or all of the k-mers (i.e. plural types of k-mer state for some or all types of k-mer). The transition weightings 14 here could be between distinct origin and distinct destination k-mers, so each origin-destination pair may have plural weightings depending on the number of distinct representations of each k-mer. One of many possible interpretations of these distinct representations is that the k-mers are tagged with a label indicating some behaviour of the system that is not directly observable, for example different conformations that a polymer may adopt during translocation through a nanopore or different dynamics of translocation behaviour.

The model 13 in respect of each series of measurements 12 takes into account the properties of the measurement system 8 used to derive the series of measurements. For example, in the case of measurements of the target sequence taken by an identical measurement system 8, then the models 13 for each series of measurements may be the same. But in the case of measurements of the target sequence taken by different types of measurement system 8, then the models 13 may take into account the different signal responses of each type of measurement system 13, for example the different dependence of measurements on the different types of k-mer.

In the case that the measurements are of a sequence having a predetermined relationship with the target sequence, the model 13 also takes into account that relationship, so as to relate the measurements in respect of polymers in the measured sequence to the corresponding polymers in the target sequence. For example, in the case of measurements of a sequence that correspond to the target sequence by being complementary to the target sequence, then, compared to a model for the target sequence, the model 13 is the same except modified to apply to the complementary k-mers. For example, where the model 13 comprises transition weightings 14 and emission weightings 15 as described above, the transition weightings 14 represent the same chances of possible transitions between different types of k-mer state, but applied to the complementary k-mer states, and the emission weightings 15 represent the same chances of observing given values of measurements but applied to the complementary k-mer states.

Training of an individual model 13 for any given type of measurement system 8, that is derivation of the emission weightings 15 and transition weightings 14 in the case that these are not predefined, may be performed by taking measurements from known polymers and using training techniques that are appropriate for the type of model 13. By way of example, WO-2013/041878 describes two examples of training methods for a model 13 that is an HMM in respect of a measurement system 8 comprising a nanopore used to measure a polynucleotide, the first method using static DNA strands held at a particular position within the nanopore by a biotin/streptavidin system and the second method using measurements from DNA strands translocated through the nanopore and estimating the emission weightings by exploiting a similar probabilistic framework to that described for k-mer estimation.

Before reverting to the method of generating an estimate of a target sequence of polymer units shown in FIG. 1 using plural series of measurements 12, there will now be described a method shown in FIG. 8 of generating an estimate of the measured sequence of polymer units 23 from a single series of measurements 12 by applying the model 13 in respect of that series of measurements 12. This is useful by way of background, and parts of this method are applied in some alternatives of the method of FIG. 1 described further below.

In an estimation step T1, there is generated an initial series of estimates of k-mer states 20 in respect of each measurement by applying the model 13 of the series of measurements 12 to the series of measurements 12 itself. For each measurement, this initial series of estimates of k-mer states 20 comprises, in respect of each measurement, weightings for each possible type of k-mer state that the k-mer state from which the measurement is observed may be. Thus, each estimate of the initial series of estimates of k-mer states 20 provides information on all possible types of k-mer state, including k-mer states other than the most likely one, rather than selecting or calling a single k-mer state. This estimation step T1 may be performed using techniques applicable to the nature of the model 13. This may be based on the likelihood predicted by the model 13 of the series of measurements 12 being produced by different sequences of k-mer states. When using a model 13 that is probabilistic, the weightings may similarly be probabilities, although that is not essential.

The analytical technique applied in the estimation step T1 may take a variety of forms that are suitable to the model 13. For example in the case that the model 13 is an HMM, the analytical technique may be a known algorithm for solving the HMM, for example the Forwards-Backwards algorithm, which is well known in the art. Such algorithms in general avoid a brute force calculation of the likelihood of all possible paths through the sequence of states, and instead identify state sequences using a simplified method based on the likelihood.

In a k-mer calling step T2, the initial series of estimates of k-mer states 20 in respect of each measurement is analysed to derive a series of discrete estimated k-mer states 21 in respect of each measurement. This involves selecting or calling a single k-mer state in respect of each measurement. Thus, the series of discrete estimated k-mer states 21 may be considered as a series of estimates of a k-mer state in respect of each measurement that each comprises a single estimate deemed to be the most likely k-mer state. The k-mer calling step T2 may take a variety of forms that are suitable to the model 13, and that is consistent with the approach in the estimation step T1.

As an example where the estimation step T1 uses the Forwards-Backwards algorithm, the initial series of estimates of k-mer states 20 is derived based on the likelihood predicted by the model of the series of measurements being observed from the individual k-mer states. The Forwards-Backwards algorithm is well known in the art. For the forwards part, the total likelihood of all sequences ending in a given k-mer state is calculated recursively forwards from the first to the last measurement using the transition and emission weightings. The backwards part works in a similar manner but from the last measurement through to the first. These forwards and backwards probabilities are combined and along with the total likelihood of the data to calculate the probability of each measurement being from different types of k-mer state, as the initial series of estimates of k-mer states 20.

In this example, in the k-mer calling step T2, the series of discrete estimated k-mer states 21 in respect of each measurement is derived from the Forwards-Backwards probabilities. This is based on the likelihood associated with each individual k-mer state. One simple approach is to take the most likely k-mer state in respect of each measurement, because the Forwards-Backwards probabilities indicate the relative likelihood of k-mer states at each measurement.

In another alternative, the k-mer calling step T2 may derive the initial series of estimates of k-mer states 20 in respect of each measurement by estimating the overall sequence, or plural overall sequences, based on the likelihood predicted by the model of the series of measurements being produced by overall sequences of k-mer states.

As an alternative the estimation step T1 may generate the series of discrete estimated k-mer states 21 directly, in which case the k-mer calling step T2 is unnecessary and is not performed. In this alternative, the analytical technique applied in the estimation step T1 may take a variety of forms that are suitable to the model 13.

As an example in the case that the model 13 is an HMM, the analytical technique may be a known algorithm for solving the HMM, for example the Viterbi algorithm which is well known in the art. In that case, the series of discrete estimated k-mer states 21 is derived based on the likelihood predicted by the model 13 of the series of measurements 12 being produced by overall sequences of k-mer states.

As another example in the in the case that the general model 30 is an HMM, the analytical technique may be of the type disclosed in Fariselli et al., “The posterior-Viterbi: a new decoding algorithm for hidden Markov models”, Department of Bilogy, University of Casadio, archived in Cornell University, submitted 4 Jan. 2005. In this method, a posterior matrix (representing the probabilities that the measurements are observed from each k-mer state) and obtain a consistent path, being a path where neighbouring k-mer states are biased towards overlapping, rather than simply choosing the most likely k-mer state per event. In essence, this allows recovery of the same information as obtained directly from application of the Viterbi algorithm.

The above techniques applied in the estimation step T1 and k-mer calling step T2 are not limitative. There are many ways to utilise the model 13 using a probabilistic or other analytical technique. The process of generating the initial series of estimates of k-mer states 20 and the series of discrete estimated k-mer states 21 can be tailored to a specific application. It may be that only one of the initial series of estimates of k-mer states 20 and the series of discrete estimated k-mer states 21 are produced as a series of estimates. It is not necessary to derive the series of discrete estimated k-mer states 21 as “hard” k-mer calls. There can be considered all k-mer state sequences, or a sub-set of likely k-mer state sequences. There can be considered k-mer states or sets of k-mer states either associated with k-mer state sequences or considered independently of particular k-mer state sequences, for example a weighted sum over all k-mer state sequences.

The above description is given in terms of a model 13 that is a HMM in which the transition weightings 14 and emission weightings 15 are probabilities and the estimation step T1 and k-mer calling step T2 use a probabilistic technique that refers to the model 13. However, it is alternatively possible for the model 13 to use a framework in which the transition weightings 14 and/or the emission weightings 15 are not probabilities but represent the chances of transitions or measurements in some other way. In this case, the estimation step T1 and k-mer calling step T2 may use an analytical technique other than a probabilistic technique that is based on the likelihood predicted by the model 13 of the series of measurements being produced by sequences of polymer units. The analytical technique may explicitly use a likelihood function, but in general this is not essential. Thus in the context of the present disclosure, the term “likelihood” is used in a general sense of taking account of the chance of the series of measurements being produced by sequences of polymer units, without requiring calculation or use of a formal likelihood function.

For example, the transition weightings 14 and/or the emission weightings 15 may be represented by costs (or distances) that represent the chances of transitions or emissions, but are not probabilities and so for example are not constrained to sum to one. In this case, the estimation step T1 and k-mer calling step T2 may use an analytical technique that handles the analysis as a minimum cost path or minimum path problem, for example as seen commonly in operations research. Standard methods such as Dijkstra's algorithm, or other more efficient algorithms, can be used for solution.

In the alternative that the state detection step S2 is omitted, the estimation step T1 is applied directly to the series of raw measurements 11 in which groups of plural measurements are dependent on the same k-mer without a priori knowledge of the number of measurements in a group. In this case, very similar techniques can be applied in the estimation step T1, but with an adjustment to the model 13 as discussed above in which the transition weightings 14 are reduced from each given type of the origin k-mer state to destination k-mer states of a different type so that the sum of the transition probabilities away from any given type of the origin k-mer state to destination k-mer states of different type is less than 1, typically much less than 1 This reduction takes account of the fact that a larger number of measurements in respect of each type of k-mer state are present the series of raw measurements 11.

For example, if on average the system spends 100 measurements at the same type of k-mer state, then the probability on the diagonals in the transition matrix (representing no transition or a transition in which the origin k-mer state and destination k-mer state are the same k-mer state) will be 0.99 with 0.01 split between all the other preferred and non-preferred transitions. The set of preferred transitions may be similar to those for the case that the state detection step S2 is performed.

In the case the k is one, then the series of discrete estimated k-mer states 21 in respect of each measurement is effectively a final estimate of the measured sequence of polymer units, and so the further sequence call step T3 described below is not performed.

However, in the case that k is a plural integer, then in a sequence call step T3, the series of discrete estimated k-mer states 21 in respect of each measurement, or alternatively the initial series of estimates 20 in respect of each measurement that comprise weightings for each possible type of k-mer state, is analysed to derive a series of estimated k-mer states 22 in respect of each k-mer in an underlying sequence that corresponds with an estimate of the measured sequence of polymer units 23. This is possible in the case that k is a plural integer, because successive discrete estimated k-mer states 21 correspond to polymer units that overlap with each other. In the event of skips or stays, the series of discrete estimated k-mer states 21 in respect of each measurement may include successive estimated k-mer states that are inconsistent with an underlying sequence of polymer units. Thus, the series of discrete estimated k-mer states 21 can be analysed based on the overlaps to derive the estimate of the measured sequence of polymer units 23 and the corresponding series of estimated k-mer states 22.

The sequence call step T3 may be performed using any suitable technique. A probabilistic approach may be applied to derive the estimate of the measured sequence of polymer units 23 in accordance with the series of discrete estimated k-mer states 21, or alternatively the initial series of estimates 20.

One straightforward approach for the sequence call step T3 is to use the initial series of estimates 20 in respect of each measurement that comprise weightings for each possible type of k-mer state and to relate the estimates of the initial series of estimates 20 to polymer units in the estimate of the measured sequence of polymer units 23. In that case, each polymer unit the estimate of the measured sequence of polymer units 23 may be estimated solely from the corresponding k-mer state estimates, deriving this as being most likely k-mer state indicated thereby. The corresponding series of estimated k-mer states 22 may be derived by expanding the estimate of the measured sequence of polymer units 23.

More complicated approaches for the sequence call step T3 are to estimate each polymer unit using a combination of information from the group of estimated k-mer states that contain the given polymer unit. For each position, all the estimates of k-mer states that contain the polymer unit corresponding to that position are used. As these estimates are weighted or probabilistic, they may be combined to generate the most likely polymer unit for that position.

As the sequence call step T3 is performed using weightings or probabilistically, the sequence call step T3 may similarly weightings or probabilities for different possible types of polymer unit in the estimate 23.

In performing this sequence call step T3, the mapping between the series of discrete estimated k-mer states 21 in respect of each measurement and the series of estimated k-mer states 22 in respect of each k-mer in an underlying sequence is retained and so the series of estimated k-mer states 22 and the estimate of the measured sequence of polymer units 23 are each mapped to the series of measurements 12 themselves, albeit not necessarily in a one-to-one mapping.

This description now reverts to the method of generating an estimate of a target sequence of polymer units shown in FIG. 1 using plural series of measurements 11.

In an alignment step S3, an estimated alignment mapping 16 between the plural series of measurements 12 is derived. The alignment step S3 is derives the estimated alignment mapping 16 based on an application of the model 13 in respect of each series of measurements 12 to the measurements of that series, using techniques described further below. In the case that the measured sequences have a predetermined relationship with each other, rather than being the same sequences (for example one of the measured sequences being the target sequence and the other measured sequence having a predetermined relationship with the target sequence, such as being complementary, rather than both of the sequences being the target sequence) then alignment step S3 takes into account that relationship. That is, the measurements in respect of the polymers that are related are mapped, rather than measurements in respect of the same polymer.

The estimated alignment mapping 16 aligns each measurement of each series of measurements 12 to a measurement in the other series or to a gap in the other series. By way of example, FIG. 9 illustrates an example of an alignment mapping 16 between a first series of measurements m₁ to m₁₀ and a second series of measurements n₁ to n₉, wherein the vertical lines indicate an alignment between two measurements, or in the case of a dash between an alignment between a measurement in one series and a gap in the other series. In this example, measurements m₁ to m₃ in the first series of measurements are aligned with (mapped to), respectively, n₁ to n₃ in the second series of measurements, whereas measurements m₄ to m₅ in the first series of measurements are aligned with (mapped to) gaps in the second series of measurements, and so on.

There will now be described some different techniques deriving the estimated alignment mapping 16 between the plural series of measurements 12 that may be applied in the alignment step S3.

A first method of performing the alignment step S3 is shown in FIG. 10 and performed as follows. This method is “symmetrical” in the sense that each series of measurements 12 is processed in the same way.

In a k-mer estimation step S3-a1, each series of measurements 12 is analysed in the same manner. In particular, in respect of each series of measurements 12, a series of estimates 25 of k-mer states is derived by applying the model 13 of that series of measurements 12 to the series of measurements 12 itself. This may be done by applying the method shown in FIG. 8 in various alternative ways.

In a first alternative, the series of estimates 25 of k-mer states is the same as the initial series of estimates of k-mer states 20 in respect of each measurement that comprise weightings, in respect of each measurement, for each possible type of k-mer state, as described above. This may be achieved by the k-mer estimation step S3-a1 comprising the estimation step T1 of the method of FIG. 8.

In a second alternative, the series of estimates 25 of k-mer states is the same as the series of discrete estimated k-mer states 21 in respect of each measurement, as described above. This may be achieved by the k-mer estimation step S3-a1 comprising the estimation step T1 and the k-mer calling step T2 of the method of FIG. 8 (or just the estimation step T1 in the alternative described above that the k-mer calling step is omitted).

In a third alternative, the k-mer estimation step S3-a1 comprises the entire method of FIG. 8, so that the series of estimates 25 of k-mer states is the same as the series of estimated k-mer states 22 in respect of each k-mer in the underlying sequence that corresponds with an estimate of the measured sequence of polymer units 23 and that are mapped to the series of measurements 12 themselves, as described above.

With respect to the first type of technique, in an alignment step S3-a2, the estimated alignment mapping 16 between the series of measurements is derived by comparing the plural series of estimates 25 of k-mer states derived in the k-mer estimation step S3-a1. In the case that the measured sequences have a predetermined relationship with each other, rather than being the same sequence, then alignment step S3-a2 takes into account that relationship. That is, measurements in respect of the polymers that are related are mapped, rather than measurements in respect of the same polymer.

The alignment step S3-a2 may perform the comparison using a substitution scoring function 18, and may be a development of known alignment techniques for pairwise alignment of sequences, such as are known for DNA sequence alignment, for example a standard Needleman-Wunsch alignment algorithm.

Such known DNA sequence alignment methods work reasonably well for aligning the two sequences of types of nucleotide together when the estimates of individual nucleotides are themselves very accurate. However, it is still quite easy in cases where there is an error in one of the base-calls for this to result in an off-by-one error in the resulting alignment. As a result, while the alignment may be nearly correct, such methods are not useful for combining plural dimensional measurements, because the alignment will not be sufficient to correct the errors made in the estimates of individual nucleotides. This problem is reduced by the alignment in k-mer state space. This is useful because each observed measurement is dependent not on a single base, but on an entire k-mer which may be thought of as a subsequence of some length, all of the polymer units of the k-mer contributing to the current level observed for that event.

The substitution scoring function 18 is a function that provides a score in respect of different possible alignment between the measurements of the respective series of measurements 12. The substitution scoring function 18 may comprise a combination, for example a sum, of (a) substitution scores and (b) gap penalties for the possible alignment.

The substitution scores are generated in respect of measurements in the plural series of measurements 12 that that the possible alignment aligns together. By way of illustration, in respect of the possible alignment shown in the example of FIG. 9, the scoring function 18 provides substitution scores in respect of the measurements m₁ to m₃, m₆ to m₉ and m₁₀ in the first series and their aligned measurements n₁ to n₃, n₄ to n₇ and n₉ in the second series.

The substitution scores represent the likelihood, optionally a probability, that individual estimates in the series of estimates 25 of k-mer states in respect of the measurements in the series of measurement 12 are derived from corresponding polymer units in the respective sequences from which the series of measurements 12 derive, or in other words corresponding to the likelihood of any underlying polymer unit producing those specific measurements in the series. The purpose of the substitution scores is to make it more expensive to align a symbol in one sequence to a different symbol in the other sequence.

In the first alternative mentioned above that the series of estimates 25 of k-mer states is the initial series of estimates of k-mer states 20 in respect of each measurement that comprise weightings for each possible type of k-mer state, then the substitution scores may be derived from weightings for each possible type of k-mer state. This effectively uses a weighting or probability distribution for different possible types of k-mer state in respect of each measurement, rather than just a single result. Instead of trying to align sequences of nucleotides, sequences of weightings or probability functions are aligned. This allows the substitution scores to take account of probabilities for different types of k-mer state, that may still have substantial weight associated with the correct type of k-mer state and may therefore impart useful information even in the event of imperfect measurements.

In a simple form, the substitution scores may be derived from a sum of the products of the probabilities of each type of k-mer state, for example being equal to that sum or to the natural log of that sum. In the limit that the two weighting or probability distributions are identical, this will give a maximal score. In the extreme case that the two distributions have no overlap at all, the score will be zero. In this way, the relative likelihood associated with the kinds of substitution mistakes that may have been made by the base-caller are automatically factored into the scoring function. Various normalization strategies can also be used.

In the second alternative mentioned above that the series of estimates 25 of k-mer states is the series of estimates 25 of k-mer states is the series of discrete estimated k-mer states 21 in respect of each measurement, then the substitution scores may be calculated in a similar manner, but simplified in that instead of a weighting for each type of k-mer state, there is a single estimated k-mer state in respect of each measurement.

Mathematically this can be represented as follows. for the example of two series of measurements 12 in the case that the weightings for each type of k-mer state are probabilities.

Firstly, where N is the number of possible types of k-mer states, Xᵢ=(X₁, X₂, . . . , X_{N}) is taken to represent the set of weightings Xₙ for the i-th measurement in the first series of measurements 12 in respect of the n-th type of k-mer state, and Yⱼ=(Y₁, Y₂, . . . , Y_{N}) is taken to represent the set of weightings Yₙ for the j-th measurement in the second series of measurements 12 in respect of the n-th type of k-mer state.

The level of descriptiveness of the sets of weightings Xᵢ and Yⱼ may vary. As an example where the sets of weightings Xᵢ and Yⱼ are descriptive, in the first alternative mentioned above that the series of estimates 25 of k-mer states is the initial series of estimates of k-mer states 20 in respect of each measurement that comprise weightings for each possible type of k-mer state, then the weightings Xₙ and Yₙ are those weightings.

As an example where the sets of weightings Xᵢ and Yⱼ are not descriptive, in the case that the second alternative mentioned above that the series of estimates 25 of k-mer states is the series of discrete estimated k-mer states 21 in respect of each measurement, then the weightings Xₙ and Yₙ have a binary value of 1 for the type of k-mer state that has been estimated or of 0 for the other types of k-mer state.

Secondly, there is used a relative likelihood matrix M comprising components representing the relative likelihood of an actual k-mer state being both estimated as being the n-th type of k-mer state from the first series of measurements 12 and estimated as being the m-th type of k-mer state from the second series of measurements 12. In other words, the relative likelihood matrix M which is a transformation that represents information about how easily individual k-mer states should be able to align to one another. This relative likelihood matrix M provides a score associated with every possible matching of symbols. The diagonal of this relative likelihood matrix M (corresponding to pairing of a symbol with itself) will have the highest score, with the scores of the other positions reflecting how frequently such pairings are expected. The relative likelihood matrix M is therefore stored data representing these relative likelihoods in respect of each possible combination of a type of k-mer state estimated from the first series of measurements and a type of k-mer state estimated from the second series of measurements.

Again, the level of descriptiveness of the relative likelihood matrix M may vary.

As an example where it is not descriptive, the relative likelihood matrix M may simply be the identity matrix. In that case, the mismatch scores will depend entirely on the sets of weightings Xᵢ and Yⱼ. This may be applicable in the case that those sets of weightings Xᵢ and Yⱼ are descriptive, for example in the first alternative mentioned above that the series of estimates 25 of k-mer states is the initial series of estimates of k-mer states 20 in respect of each measurement that comprise weightings for each possible type of k-mer state.

As an example where the relative likelihood matrix M is descriptive, the relative likelihood matrix M may include non-zero values representing the actual relative likelihoods for all possible types of k-mer states. This may be applicable in the case that the sets of weightings Xᵢ and Yⱼ are not descriptive, for example in the case that the series of estimates 25 of k-mer states is the series of discrete estimated k-mer states 21 in respect of each measurement, so that the mismatch scores depend entirely on that matrix.

With this representation of the sets of weightings Xᵢ and Yⱼ and the relative likelihood matrix M, the substitution score S_{i,j} for an alignment between the i-th measurement of the first series and the j-th measurement of the second series may be given by either of the following two equations (1) and (2):

S_{i,j}=(XᵢMYⱼ^{T}) (1)

S_{i,j}=ln(XᵢMYⱼ^{T}) (2)

It should be noted that in the case that the relative likelihood matrix M is the identity matrix, then these equations (1) and (2) collapse down to the following equations (3) and (4), respectively:

S_{i,j}=(XᵢYⱼ^{T}) (3)

S_{i,j}=ln(XᵢYⱼ^{T}) (4)

Equally, in the case that the series of estimates 25 of k-mer states is the series of discrete estimated k-mer states 21 in respect of each measurement, then due to the binary nature of the sets of weightings Xᵢ and Yⱼ, equations (1) and (2) have the result of selecting a single one of the components being the relative likelihood of an actual k-mer state being estimated as being the discrete estimate of index n that is actually estimated from the first series of measurements 12 (as represented by the n-th type of k-mer state that has a binary value 1) and estimated as being the discrete estimate of index m that is estimated from the same measurement in the second series of measurements 12 (as represented by the m-th type of k-mer state that has a binary value 1).

The relative likelihood matrix M may be derived using techniques known to the art based on based on knowledge of the physical source of such mismatches, for example based on actual experimental data or on data simulated from a model that has been trained on experimental data. In general terms this may be achieved by counting how often one type of k-mer state is aligned to another in a set of exemplar alignments. Such alignments may be created by an expert or generated automatically. One way to automatically create a set of exemplar alignments is to take both series of measurements from a known sequence and align each series of measurements to the known sequence to get an implicit alignment between the measurements and hence k-mers. Possible ways in which each series of measurements may be aligned to the known sequence include those disclosed in WO-13121224. The relative likelihood matrix M may itself be dependent upon the state of the system, for example an external environmental factor such as ambient temperature.

An example of a process for calculating the relative likelihood matrix M is as follows.

A large set of experimental or simulated series of measurements is used providing many contexts for each type of k-mer state. For each series of measurements the types of k-mer state are estimated. Then a count is made of how often each actual type of k-mer state is estimates as each of the possible types of k-mer state. This provides a two-dimensional matrix of counts of possible types of k-mer state in the estimates against actual possible types of k-mer state. In general, each entry in the matrix could have a value although in practice some, often many, entries will be zero, because some types of k-mer are simply never miscalled as certain other types of k-mer state.

Next, this matrix is normalised so that, for each actual type of k-mer state (row), the matrix indicates the fraction of the time it is called as each possible type of k-mer state (column).

This is done in respect of the model of each series of measurements. In the case that the measurements are of a sequence having a predetermined relationship with the target sequence, the matrix also takes into account that relationship, so as to relate the measurements in respect of polymers in the measured sequence to the corresponding polymers in the target sequence. For example, in the case of measurements of a sequence that correspond to the target sequence by being complementary to the target sequence, then this is achieved by the matrix being organised so that each row corresponds not to the actual type of k-mer in the complementary sequence, but instead to the corresponding type of k-mer in the target sequence.

This means that the columns of the matrices in respect of each series of measurements indicate the relative likelihood of a type of k-mer state that has been estimated having been derived from different actual types of k-mer state. Thus, each entry in the relative likelihood matrix M is derived by taking the dot product of the column in the matrix in respect of one series of measurements corresponding to a first respective type of k-mer state and the column in the matrix in respect of the other series of measurements corresponding to a second respective type of k-mer state. To give an example where k is three, for an event estimated as the type of k-mer state ‘CTC’ from the first series of measurements, and an event an event estimated as the type of k-mer state ‘GCG’ from the second series of measurements, the column of the first matrix corresponding to ‘CTC’, gives the relative likelihood associated with each actual type of k-mer state, and the column of the second matrix corresponding to ‘GCG’ gives the relative likelihood associated with each actual k-mer state for that observation. Thus, the dot-product of these two columns gives us an overall likelihood that any actual k-mer state would produce the pair of estimated states ‘CTC’ and ‘GCG’. Of course, such dot-products may be derived simply by multiplying one matrix by the transpose of the other matrix. The resulting matrix is the relative likelihood matrix M.

As these calculations are performed in advance the derivation of the substitution score S_{i,j} may be performed by a simple lookup. Similarly, where the substitution score S_{i,j} is a natural logarithm in accordance with equation (2), the natural logarithms of the entries in the relative likelihood matrix M may be calculated in advance.

The relative likelihood matrix used for the alignment may be chosen from a number of substitution matrices M₁, M₂, . . . , Mₙ, or a functor of many such matrices M=fₐᵤₓ(M₁, M₂, . . . , Mₙ), the choice or blend being dependent on auxiliary information “aux” about the system. For example, each member of the set of substitution matrices may be appropriate for a different known temperature, and the actual substitution matrix used may be an interpolation based on the measured temperature of the system. As an example of a simple interpolation case, M=ai Mi+aj Mj, where ai and aj depend on the measured temperature t and the known temperatures for Mi and Mj, ti and tj respectively being the closest temperatures to t satisfying ti<=t<=tj, and wherein ai=(tj−t)/(tj−ti) and aj=(t−ti)/(tj−ti).

Additionally, one may find it useful to reduce down the k-mer state space by combining states together to form fewer states. For example, the method is performed with a 5-mer model 13, for use in the feature alignment analysis step S4, the probabilistic estimate 16 may be made in the alignment step S3 in a 3-mer state-space. The reduction of 5-mers to 3-mers, namely the reduction of 1024 possible states to 64 states considerably reduces the amount of computation necessary to form the substitution scores while retaining much of the information relevant to the alignment.

The example transformation from 5-mers to 3-mers is a direct mapping between two state-spaces, where every 5-mer corresponds to a single 3-mer state. A reduced state-space need not be a direct mapping of the k-mer space and more generally, any new state-space can be defined where there is a transformation from one or more k-mer states. The transformation need not uniquely associate k-mer states to states in the new space and each new state could be an abstract state representing a weighting of k-mer states. For example, the reduction from 5mers to 3mers could be represented by a linear transformation on the k-mer probabilities with unit contribution from each 5-mer that contains the specific 3-mer. Other types of reduction could be applied. For example a more general linear transformation could be used, where the scores for each abstract state are linear weightings of the k-mer probabilities. The transformation used need not be the same for the two series of measurements but we find using the same transformation to be satisfactory. A suitable pair of linear transformations from k-mer states into n abstract states and an appropriately reduced substitution matrix may be obtained by performing a Single Value Decomposition and keeping only those components with the n largest singular values. The remaining singular values form a diagonal substitution matrix between the abstract states. Other methods known to the art for forming a lower dimension approximation of a matrix may also be used.

The weightings Xᵢ and Yⱼ may be augmented with additional elements representing additional information about the measurement, which may be non-probabilistic, such as a score representing whether the measurement is to be trusted or a function of some state of the system, for example an external environmental factor such as ambient temperature. The probabilities may also be augmented with additional elements representing nonlinear functions of other elements. This allows for generic feature alignment where the “features” can be whatever set of characteristics of the k-mers that is found to be most useful for deciding whether or not measurements should be aligned to each other.

In the third alternative mentioned above that the series of estimates 25 of k-mer states is the series of estimates 25 of k-mer states is the series of estimated k-mer states 22 in respect of each k-mer in the underlying sequence that corresponds with an estimate of the measured sequence of polymer units 23, then the substitution scores may be calculated in the same manner as the second alternative (i.e. similar to the first alternative but simplified in that instead of a weighting for each type of k-mer state, there is a single estimated k-mer state in respect of each measurement), but taking account of the mapping between the series of estimated k-mer states 22 in respect of each k-mer in the underlying sequence and the series of measurements 12 themselves. Thus, the substitution score S_{i,j} for an alignment between the i-th measurement of the first series of measurements 12 and the j-th measurement of the second series of measurements 12 may be calculated in the same manner as the example described above except that probabilities Xₙ and Yₙ in the sets Xᵢ=(X₁, X₂, . . . , X_{N}) and Y=(Y₁, Y₂, . . . , Y_{N}) have a value of 1 for the estimated types of k-mer state in the series of estimates 25 of k-mer states that map to the i-th and j-th measurement in the first and second series of measurements and a value of 0 for the other types of k-mer state.

The gap penalties are generated in respect of measurements in the plural series of measurements 12 that that the possible alignment aligns to a gap in the other series. By way of illustration, in the example of FIG. 9, gap penalties are provided in respect of measurements m₄, and m₅ in the first series and measurement n₈ in the second series.

The gap penalties represent the likelihood that the other series does not include a corresponding measurement. Gap penalties are assessed in the same way they would be for a conventional alignment in the polymer unit space.

As with the substitution scores, assessment of the appropriate gap penalties may make use of information about what the physical sources of such gaps in the alignment are. This means that the gap penalty may reflect how frequently gaps are expected and how the size of the gap penalty should scale with the length of the gap. At one extreme, the penalty could be proportional to the number of k-mer states, which has the effect of causing it to be equally likely to have a single long gap, or many small gaps adding up to the same length. This makes sense in situations where gaps in the alignment are due to errors in the derivation of the series of estimates 25 of k-mer states, rather than being due to actual differences in the physical sequences the measurements were taken. On the other hand, in many types of measurement system 8, insertions or deletions tend to come in groups, in which case a large penalty may be imposed for opening a gap, and then a much smaller penalty for continuing the gap.

The gap penalty may be consistent with the likelihood predicted by models 13 in respect of the series of measurements 12 that the other series does not include a corresponding measurement. For example, the gap penalty may be derived from a ratio of the likelihood or probability P_{gap} predicted by model 13 that a measurement is aligned to a gap in the other series to the likelihood of probability P_{nogap} predicted by model that a measurement is not aligned to a measurement in the other series for example being equal to that ratio (for example in the case that the substitution score S_{i,j} is given by equations (1) or (3)) or to the natural log of that ratio (for example in the case that the substitution score S_{i,j} is given by equations (2) or (4)). Expressing this mathematically, the gap penalty S_{gap} may be given by either of the following two equations (5) and (6):

$$\begin{matrix}{S}_{gap} = ( \frac{{P}_{gap}}{{P}_{no gap}} ) & ( 5 ) \\ {S}_{gap} = ln ( \frac{{P}_{gap}}{{P}_{no gap}} ) & ( 6 )\end{matrix}$$

The gap penalty can also be determined empirically using measurements taken from known sequences. In such a case, as a result of the correct alignment being known, it is straightforward to estimate how often, on average, measurements from one series align to gaps in another series.

By way of example, the following considerations apply to a scoring function for a template-complement method based on measurements of a target sequence and a sequence complementary to the target sequence from the same polynucleotide.

In this example, any differences between the measured template and complement sequences, after taking into account the reverse-complement nature of the complement sequence will be due to errors made in the analysis of the signals, rather than due to the actual polynucleotide being different. This can be due to many factors, such as artefacts in the signal, bases being missed due to the speed of translocation of the polynucleotide, or ambiguity in the model 13, meaning that multiple polynucleotide sequences can result in identical or nearly identical measurements.

With respect to substitution scores in this example, the relative frequency with which various nucleotides tend to be miscalled as other nucleotides can be empirically determined by sequencing large amounts of known polynucleotide sequences. An optimal substitution matrix M can then be derived from those results.

With respect to gap penalties in this example, in most situations using a fixed gap penalty that applies equally to each nucleotide that aligns to a gap is sensible. In some cases, if it is observed that missed measurements of k-mers or excess measurements due to artefacts tend to occur in clusters, then a gap penalty that is a function of length can be used instead, and the specific function to use can again be determined empirically by sequencing known nucleotide sequences.

The alignment step S3-a2 uses the substitution scoring function 18 by deriving the estimated alignment mapping 16 as the possible alignment that maximises the scoring function. Thus, in the example that the estimated alignment mapping 16 is that shown in FIG. 9, this alignment mapping has a higher scoring function than other possible alignment mappings between the series of measurements 12.

Whilst the alignment analysis could in principle apply a brute force technique which considers every possible alignment mapping between the series of measurements 12, that would be computationally expensive. Accordingly, the alignment analysis may derive the possible alignment that maximises the scoring function using a dynamic programming technique. A range of suitable dynamic programming techniques are used in known alignment techniques for pairwise alignment of sequences, such as DNA sequence alignment and may be applied here. A non-limitative examples of a suitable dynamic programming technique is to use a standard Needleman-Wunsch alignment algorithm.

Although the above methods are described with reference to an example using two series of measurements 12, that example may be generalised to find the estimated alignment mapping 16 between three or more series of measurements 12 simply by increasing the dimensionality of the comparison and consideration of possible alignments between the series of measurements, in particular increasing the dimensionality of the scoring function 18.

This first method of performing the alignment step S3 shown in FIG. 10 that is symmetrical with respect to the series of measurements 12 derives the estimated alignment mapping 16 in the space of the k-mer states by using the model 13 of each series of measurements 12 to convert the each series of measurements 12 into a series of estimates 25 of k-mer states. This allows the estimation of the alignment mapping 16 to preserve much of the information contained in the original measurements, while at the same time abstracting from the measurement space to the k-mer space that allows for direct correspondence of data. Since the k-mer states can be informed by every measurement in the plural series, they can be much more informative about the type of k-mer state of each k-mer in the target sequence than the corresponding raw measurement.

A second method of performing the alignment step S3 that is applicable when k is a plural integer is shown in FIG. 11 and performed as follows. This method is “asymmetrical” in the sense that that each series of measurements 12 is handled in a different way. To distinguish the series of measurements 12 in the following discussion, labels 12a and 12b are given to a first series of measurements 12a and the other series of measurements 12b. In this example there is only one other series of measurements 12b, but the method may be applied in a similar manner to plural other series of measurements.

The first series of measurements 12a is analysed as follows.

In a k-mer estimation step S3-b1, the model 13 of the first series of measurements 12a is applied to the series of measurements 12 itself to derive a series of estimated k-mer states 26. This step may use the method shown in FIG. 8.

The series of estimated k-mer states 26 may be estimated k-mer states in respect of each measurement. In that case, the k-mer estimation step S3-b1 may comprise the estimation step T1 and the k-mer calling step T2 of the method of FIG. 8, so that the series of estimates of estimated k-mer states 26 is the same as the series of discrete estimated k-mer states 21 in respect of each measurement, as described above.

Alternatively, the series of estimated k-mer states 26 may be estimated k-mer states in respect of each k-mer state in an underlying sequence that corresponds with an estimate of the measured sequence of polymer units 23. In that case, the k-mer estimation step S3-b1 may comprise the estimation step T1, the k-mer calling step T2 and the sequence call step T3 of the method of FIG. 8 so that the series of estimated k-mer states 26 is the same as a series of estimated k-mer states 22, as described above. In that case, the estimated k-mer states of the series of estimated k-mer states 26 are each mapped to the measurements of the first series of measurements 12, albeit not necessarily in a one-to-one mapping.

In the case that the measured sequences have a predetermined relationship with each other, rather than being the same sequence, then in k-mer estimation step S3-b1 the series of estimated k-mer states derived by application of the model 13 are transformed taking into account that relationship to derive the estimated k-mer states 26. That is, each estimated k-mer state derived by application of the model 13 is transformed into the related k-mer state of the other series of measurements 12b. For example, in the case that the predetermined relationship is that the first series of measurements 12a and the other series of measurements 12b are complementary, then each estimated k-mer state derived by application of the model 13 is transformed into the complementary state which is taken as the estimated k-mer state 26. In this manner, the derivation in alignment step S3-b3 described below of the estimated alignment mapping 16 between the plural series of measurements 12 takes into account the predetermined relationship.

In a model generation step S3-b2, a reference model 30 is derived from the series of estimated k-mer states 26.

The reference model 30 is a model of the measurement of the series of estimated k-mer states 26 in the measurement system 8. The reference model 30 may be considered as an adaption of the model 13 described above to model the measurements that are obtained specifically when the series of estimated k-mer states 26 is measured. Thus, the reference model 30 treats the measurements as observations of the series of estimated k-mer states 26. As such, the reference model 30 has the same form as the model 13 described above, in particular comprising transition weightings 31 and emission weightings 32 as will now be described.

The transition weightings 31 represent transitions between the k-mer states 26 of the model 30. Similarly, each k-mer states 26 is one of the possible types of k-mer state. Thus, the series of k-mer states 26 of the reference model 30 may be considered as one particular path through the k-mer states of the model 13.

This is illustrated with reference to the state diagram of FIG. 12 which shows an example of three successive k-mer states 26 in the series of estimated k-mer states 26. In this example, k is three and the reference sequence of polymer units includes successive polymer units labelled A, A, C, G, T. (although of course those specific types of the k-mer states 26 are not limitative). Accordingly, the successive k-mer states 26 of the model 30 corresponding to those polymer units are of types AAC, ACG, CGT which correspond to a measured sequence of polymer units AACGT.

The state diagram of FIG. 13 illustrates transitions between the k-mer states 26 of the model 30, that are represented by the transition weightings 31. In this example, only forwards progress through the k-mer states 26 of the model 30 is allowed (although in general backwards progression could additionally be allowed). Three different types of transition 34, 35 and 36 are illustrates as follows.

From each given k-mer state 26 in the series of k-mer states 26, a transition 34 to the next k-mer state 26 is allowed. This models the likelihood of successive measurements in the series of measurements 12 being taken from successive k-mers the series of k-mer states 26. As the analysis is of the series of measurements 12 that have been processed to identify successive groups of raw measurements and to derive a series of measurements 12 consisting of a predetermined number of measurements in respect of each identified group, the transition weightings 31 represent this transition 34 as having a relatively high likelihood.

From each given k-mer state 26 in the series of k-mer states 26, a transition 35 to the same k-mer state 26 is allowed. This models the likelihood of successive measurements in the series of measurements 12 being taken from the same k-mers 26 of t the series of k-mer states 26. This may be referred to as a “stay”. As the analysis is of the series of measurements 12 that have been processed to identify successive groups of raw measurements and to derive a series of measurements 12 consisting of a predetermined number of measurements in respect of each identified group, the transition weightings 31 represent this transition 35 as having a relatively low likelihood compared to the transition 34.

From each given k-mer state 26 in the series of k-mer states 26, a transition 36 to the subsequent k-mer states 26 beyond the next k-mer state 26 is allowed. This models the likelihood of no measurement being taken from the next k-mer state, so that successive measurements in the series of measurements 12 being taken from k-mers of the reference sequence of polymer units that are separated. This may be referred to as a “skip”. As the analysis is of the series of measurements 12 that have been processed to identify successive groups of raw measurements and to derive a series of measurements 12 consisting of a predetermined number of measurements in respect of each identified group, the transition weightings 31 represent this transition 36 as having a relatively low likelihood compared to the transition 34.

The level of the transition weightings 31 representing the transitions 35 and 36 for skips and stays, relative to the level of the transition weightings 31 representing the transitions 34, may be derived in the same manner as the transition weightings 31 for skips and stays in the model 13, as described above.

In the alternative that the state detection step S2 is omitted so that the further analysis is performed on the series of raw measurements 11 themselves, instead of the series of measurements 12, then the transition weightings 31 are similar but are adapted to increase the likelihood of the transition 35 representing a skip to represent the likelihood of successive measurements being taken from the same k-mer. The level of the transition weightings 31 for the transition 35 are dependent on the number of measurements expected to be taken from any given k-mer and may be determined by experiment for the particular measurement system 8 that is used.

Emission weightings 32 are provided in respect of each k-mer state 26. The emission weightings 32 are weightings for different measurements being taken when the k-mer state 26 is observed. The emission weightings 32 are therefore dependent on the type of the k-mer state 26 in question. In particular, the emission weightings 32 for a k-mer state 26 of any given type are the same as the emission weightings 32 for that type of k-mer state in the model 13 as described above.

In the model generation step S3-b2, a reference model 30 is derived from the series of estimated k-mer states 26 as follows.

The transition weightings 31 are derived for transitions between the series of estimated k-mer states 26 derived in the k-mer estimation step S3-b1. The transition weightings 21 take the form described above, defined with respect to the series of estimated k-mer states 26.

The emission weightings 32 are derived for each k-mer state 26 in series of estimated k-mer states 26 derived in the k-mer estimation step S3-b1, by selecting the emission weightings 32 from the weightings of the model 13 of the other series of measurements 12b according to the type of the k-mer state 26 Thus, the emission weightings 32 for each type of k-mer state 26 in the reference model 30 are the same as the emission weightings for that type of k-mer state 13 in the model 13 described above.

In an alignment step S3-b3, the estimated alignment mapping 16 between the plural series of measurements 12 is derived by applying the reference model 30 to the other series of measurements 12b.

As a result of the form of the reference model 30, in particular the representation of transitions between the reference series of k-mer states 26, the application of the reference model 30 intrinsically derives the estimated alignment mapping 16 between the plural series of measurements 12. This may be understood as follows. As the model 13 described above represents transitions between the possible types of k-mer state, the application of the model 13 provides estimates of the type of k-mer state from which each measurement is observed, i.e. the initial series of estimates of k-mer states 20 and the discrete estimated k-mer states 21 which are each a form of estimate of the type of the k-mer state from which each measurement is observed. As the reference model 30 represents transitions between the reference series of k-mer states 26, the application of the reference model 30 instead estimates the k-mer state 26 of the reference series of k-mer states 26 from which each measurement is observed, which is an alignment mapping between the plural series of measurements 12.

In the case that the measured sequences have a predetermined relationship with each other, rather than being the same sequence, then the predetermined relationship is taken into account by the transformation of the series of estimated k-mer states derived by application of the model 13 that is performed in k-mer estimation step S3-b1 as described above.

Depending on the method applied, the form of estimated alignment mapping 16 may vary, as follows.

As noted above, the analytical technique applied in the alignment step S3-b3 may take a variety of forms that are suitable for the form of the reference model 20. For example in the case that the reference model 20 is an HMM, the analytical technique may be a known algorithm for solving the HMM, for example the Forwards-Backwards algorithm or the Viterbi algorithm, which is well known in the art. Such algorithms in general avoid a brute force calculation of the likelihood of all possible paths through the sequence of states, and instead identify state sequences using a simplified method based on the likelihood.

With some techniques applied in the alignment step S3-b3, the estimated alignment mapping 16 comprises, for each measurement 12b in the series, weightings in respect of different k-mer states 26 in the estimated series of k-mer states 26. For example, such an alignment mapping may be represented by M_{i,j} where the index i labels the measurements of the other series of measurements 12b and the index j labels the k-mer states in the estimated series of k-mer states 26, and so where there are K k-mer states the values M_{i,1} to M_{i,K} represents the weightings in for the i-th measurement in respect of each k-mer state 26. In this case, the estimated alignment mapping 16 does not represent a single k-mer state 26 as being mapped to each measurement, but instead provides weightings for different possible k-mer states 26 being so mapped to each measurement.

As an example in the case that the reference model 30 is an HMM, the derived estimate may be of this type when the analytical technique applied is the Forwards-Backwards algorithm as described above. In the Forwards-Backwards algorithm, the total likelihood of all sequences ending in a given k-mer state is calculated recursively in for forwards and backwards directions using the transition and emission weightings. These forwards and backwards probabilities are combined along with the total likelihood of the data to calculate the probability of each measurement being from a given k-mer state. This matrix of probabilities termed the posterior matrix is the estimate 13 of the alignment mapping.

With other techniques applied in the alignment step S3-b3, the estimated alignment mapping 16 comprises, for each measurement in the other series of measurements, a discrete estimate of a k-mer state 26 in the series of estimated k-mer states 26. For example, such an alignment mapping may be represented by M where the index i labels the measurements and M can take the values 1 to K indicating the K k-mer states. In this case, the estimate 13 represents a single k-mer state 23 as being mapped to each measurement.

As an example in the case that the reference model 30 is an HMM, the derived estimate may be of this type when the analytical technique applied is the Viterbi algorithm as described above, wherein the other series of measurements 12b are analysed based on the likelihood predicted by the reference model 30 being produced by the series of k-mer states 26.

As another example in the in the case that the reference model 30 is an HMM, the analytical technique may be of the type disclosed in Fariselli et al., “The posterior-Viterbi: a new decoding algorithm for hidden Markov models”, Department of Bilogy, University of Casadio, archived in Cornell University, submitted 4 Jan. 2005, as described above.

The second method of performing the alignment step S3 contains an asymmetry in the sense that the series of measurements are treated in a different manner. The first series of measurements is used to generate an initial series of estimates of k-mer states that is used to generate a reference model, and the reference model is applied to the second series of measurements. In general, the method works irrespective of which series of measurements is used as the first series of measurements to derive the initial series of estimates of k-mer states. However, there may be situations in which advantage is achieved by selecting a particular one of the series of measurements as the first series.

In certain situations it may be the case that series of measurements are known to be of different qualities in the sense of providing in themselves different accuracy in estimation of an estimate of the target polymer sequence in a systematic manner. For example, this may result from the nature of the measurement system 8 used to generate the series of measurements. One example of a situation where this might occur is where the same measurement system 8 comprising a biological pore 1 is used to derive two series of measurements sequentially, for example when the two sequences are linked by a bridging moiety, in which case the conditions in a biological pore may vary during the translocation. In that example, it may be that one of the series of measurements is of higher data quality. For example in some situations it has been observed that the first series of polymer units that are measured in the nanopore have a higher data quality than the second series, in a systematic way. This may be due to the second series partially hybridising with the first series on the trans side of the nanopore. The fact that one of the series of measurements is of higher quality may be determined experimentally using known sequences. In other situations, the data quality may be determined from analysis of the measurements.

In the case that series of measurements are of different qualities, whether determined in advance or on the basis of analysis of the measurements, then the estimated alignment mapping is more accurate when the series of measurements of better quality is selected as the first series of measurements. The estimated alignment is best informed by utilising the series of measurements that provides the most accuracy in deriving the initial series of estimates of k-mer states, because that is closest to the k-mer states corresponding to the target polymer sequence.

This description now reverts to the method of generating an estimate of a target sequence of polymer units shown in FIG. 1. In particular, in polymer unit estimation step S4, an estimate of the target sequence of polymer units 17 is generated from the plural series of measurements 12 by applying the models 13 of each series of measurements 12. In particular, this involves performing an analysis similar to the method in respect of a single series of measurements 12 shown in FIG. 8 and described above, but using all the series of measurements 12 together in a plural dimensional model that takes account of the estimated alignment mapping 16. Thus, the polymer unit estimation step S4 treats the types of k-mer state of each model 13 as dimensions of a plural dimensional k-mer state and treats the measurements of each series of measurements 12 as plural dimensional observations of those plural dimensional k-mer states. The polymer unit estimation step S4 comprises steps that are the same as steps T1 to T3 described above but extended to this plural dimensional case.

Mathematically speaking, the extension of the method of FIG. 8 to treat the series of measurements 12 and models 13 as arranged in multiple dimensions is straightforward. The emission weightings 15 occur in plural dimensions, one dimension for each series of measurements 12. Considering model B above, conceptually the model is the same except that Tᵢ now represents a plural dimensional vector of values rather than a single value. In the case of an HMM, rather than a state emitting values from a one-dimensional probability density function go, values are emitted from a plural dimensional density function, for example in the case of measurements of two sequences, Tᵢ emits a measurement pair (tᵢₛ,tᵢₐ) where tᵢₛ is the measurement from one sequence and tᵢₐ is the measurement from the other sequence. This emitted measurement pair may contain unobserved skip states as well as real current measurements. Just as in the basic one-dimensional case, outliers and missing data, or skipped states, can be modelled. All the advantages from a one dimensional model 13 transfer to the models 13 being applied in plural dimensions.

As compared to the measurement analysis step S2, using the models in a plural dimensional manner requires consideration of an increased number of possible plural dimensional k-mer states, as occurs with the plural dimensional method disclosed in WO-2013/041878. In the absence of knowledge of the alignment between the measurements, that dimensionality would vastly increase the computational requirements, to the detriment of being able to perform the calculation quickly and/or accurately within the computational resources available practically.

However, in contrast to the plural dimensional method disclosed in WO-2013/041878, polymer unit estimation step S4 is performed using the estimated alignment mapping 16 between the series of measurements 12, in particular by constraining paths through the plural dimensional k-mer states that are considered using the derived alignment mapping 16. For example, the paths through the plural dimensional k-mer states may be constrained to be within a predetermined distance from the derived alignment mapping 16 between the plural series of measurements.

As described above for the one-dimensional case of the method of FIG. 8, the analytical technique applied may take a variety of forms that are suitable to the models 13. For example in the case that the models 13 are HMMs, the polymer unit estimation step S4 may use any known algorithm for solving the HMM, for example the Forwards Backwards algorithm or the Viterbi algorithm. Such algorithms in general avoid a brute force calculation of the likelihood of all possible paths through the sequence of states, and instead identify state sequences using a simplified method based on the likelihood. Using the estimated alignment mapping 16 as a constraint reduces the search space required to be searched, thereby reducing the computational requirements

Merely by way of example, an application of the Viterbi algorithm to series measurements 12 arranged in plural dimensions will be discussed. The Viterbi algorithm is well known in the art. For a one-dimensional HMM, the likelihood Lᵢ(k) of the most likely path ending in each possible k-mer state K is calculated for each state i moving forwards through the state sequence from the first to the last state (i=1 . . . n). Rather than considering all such paths, the estimated alignment mapping 16 is used as a constraint which reduces the number of states to be considered. The values Lᵢ(K) can be calculated using only the values Lᵢ₋₁(.) from the immediately preceding state along with the transition and emission probabilities, forming a recursion. In an plural dimensional HMM, a similar scheme may be used.

By way of example, FIG. 14 shows a grid of possible alignments between two series of measurements 12, in this example each consisting of 40 measurements although normally the number of measurements will be much higher. Each node in the grid represents a pair of measurements, one from each series. For an unconstrained, 2D method, all 1600 nodes must be processed.

In FIG. 14, the cross-hatched nodes 20 represent pairs of measurements from the two series of measurements 12 on a particular path in an example of the estimated alignment mapping 16 between the measurements of the respective series. The hatched nodes 21 represent pairs of measurements from the two series of measurements 12 that are within a predetermined distance from the derived alignment mapping 16 between the plural series of measurements 12, which predetermined distance in this example is five. The empty nodes 22 represent the remaining pairs of measurements from the two series of measurements 12 that are outside the predetermined distance from the alignment mapping 16. In this example, the measurement analysis in step S5-3 is constrained by considering only paths across the cross-hatched nodes 20 and the hatched nodes 21, that is considering pairs of measurements from each series of measurement 12 within the predetermined distance from the derived alignment mapping 16.

As long as the paths that would be produced by the unconstrained measurement analysis does not go outside of the cross-hatched nodes 20 and the hatched nodes 21, the constrained analysis will produce the same aligned base-call.

The extent of the constraint is also selected in dependence on the accuracy of the estimated alignment mapping 16. That is, as the accuracy increases, the extent of the constraint may be reduced. As discussed further below, that accuracy may be dependent on the method used to derive the estimated alignment mapping 16 and may be determined experimentally for any given method and measurement system 8 on the basis of the measurements used to train the model. The extent of the constraint is a trade-off between the number of potential mismatches which decreases with the extent of the constraint and the amount of data processing, which increases with the extent of the constraint.

In the estimation step S5, the use of the estimated alignment mapping 16 derived in generating the estimate of the target sequence of polymer units 17 allows the information from the plural series of measurements 12 to be combined in a way that corrects many of the errors that would have been made in an estimate of the target sequence derived from a single series of measurements 12.

In the simplest case, the estimate of the target sequence of polymer units 17 may be a representation that provides a single estimated type (identity) for each polymer unit. More generally, the estimate of the target sequence of polymer units 17 may be any representation of the target sequence according to some optimality criterion. For example, the estimate of the target sequence of polymer units 17 may represent, in respect of each polymer unit, a weighting or probability for the type of the most likely polymer unit, and may also represent weightings or probabilities for different types of polymer unit, optionally for all possible types of polymer unit. Alternatively, the estimate of the target sequence of polymer units 17 may comprise plural sequences, for example each including plural estimated types of one or more polymer units in part or all of the polymer.

An example applying the first method of performing the alignment step S3 in the alternative that the series of estimates 25 of k-mer states is the initial series of estimates of k-mer states 20 in respect of each measurement that comprise weightings for each possible type of k-mer state has been performed using simulated sequencing data using a 5-mer model derived from experimental data, as follows.

A 2000 base-pair chunk of the Lambda genome was selected, and event data was simulated by taking the expected current levels based on the model, plus Gaussian noise with standard deviation of 1 pA. The simulated strands are “hairpins”, meaning that in each case we have the template sequence immediately followed by the complement sequence.

To properly simulate the kinds of errors and artefacts that are commonly seen in experimental sequencing data, “skips” and “stays” were inserted into the simulated event data, a “skip” being an omitted event and a “stay” being a repetition of the previous event (but with a new realization of the noise).

To get a range of base-call qualities, simulated event data was generated for skip and stay percentages of 1%, 2%, 5%, 10%, 20%, 30%, and 40%. In each case, the skip and stay percentages were equal. Ten realizations of each condition were generated, to allow for averaging across trials.

First, the 1D base-call accuracy was computed for each event sequence. This gave base-call accuracies ranging from as low as 48% for the data with skip-stay percentages of 40%, to 78% for data with skip-stay percentages of 1%. Then, a standard base-space alignment of the template and complement base-calls was performed, and the resulting alignment used to perform a new base-call of the combined template-complement data. A k-mer state alignment of the 1D base-call posterior probabilities was also performed, using a method of the type described herein, and a new base-call of the combined template-complement data was performed using that alignment. Finally, the k-mer state alignment as a constraint for the 2D Viterbi base-calling method.

The results are shown in FIGS. 14 and 15, wherein horizontal axis is the 1D base-call quality and the vertical axis is 2D base-call quality, FIG. 15 plotting the absolute values, and FIG. 16 plotting the improvement over the 1D base-call.

The lower trace shows the results using a standard base-space alignment. For 1D call quality below 70%, the results are worse than the 1D base-call, indicating that the alignment has essentially failed. For higher 1D call values an improvement indicating a better alignment is seen. The middle trace shows the results using an alignment method in accordance with the method described herein, applied to k-mer states. Here, an improvement over the 1D base-call can be seen at around a 1D quality of 55%. For higher qualities the improvement is considerable. In all cases shown, the feature alignment results in higher accuracy than the base space alignment. Finally, the upper trace shows the results of doing a 2D Viterbi base-call using the k-mer state alignment as a constraint. Here, an additional improvement over the 1D base-call results can bee seen.

In principle, any alignment between the template and complement data can be used to constrain the 2D Viterbi method, as long as the alignment is good enough that the discrepancy between the estimated alignment and the true alignment is bounded (that is, the estimated alignment does not wander off in some sort of random-walk in an unconstrained way). In this case, the advantage of the more accurate feature alignment is a much tighter constraint on the 2D call, resulting in reduced memory and computational requirements. The memory and computational requirements for the 2D unconstrained method become particularly demanding for the alignment of polynucleotide sequences of length greater than 5 kB and longer. For the alignment of sequences of 40 kB or greater, the memory and computational requirements for the unconstrained 2D method are so great as to make it impractical.

FIG. 17 shows alignment mismatch plotted against the 1D base-call quality, illustrating the maximal mismatch (i.e. the computational complexity required in the 2D base-call) for both the base-space and the k-mer state alignments for the simulated data. The upper trace represents the base-space alignment. For 1D base-calls below 60% the alignment basically fails. Not only does this prevent the 2D base-call from improving (as seen in FIGS. 12 and 13), it also means that the alignment cannot be used to constrain the 2D Viterbi method. The lower trace shows the k-mer state alignment. This shows reasonable alignments for 1D base-calls over 50%. Additionally, for higher 1D base-calls, where the base-space alignment works, the alignment mismatch for the k-mer state alignment is much smaller. This allows the 2D Viterbi method to be constrained much more tightly, which dramatically reduces both memory and computational requirements.

An accurate estimated alignment is desirable as it allows tighter constraint on the estimation of the target sequence using the plural dimensional model. That in turn may increase reduce the processing requirements required to generate the estimate of the target sequence, which may improve the accuracy of estimation when restricted by practical limitations on processing power.

The method that provides the most accurate estimated alignment mapping may depend upon various experimental factors such as the measurement system, the sample, the ambient temperature and the k-mer type. However, the various alignment methods may be easily evaluated with respect to each other for a given type of system and experimental conditions to determine the most accurate one.

The following results demonstrate a situation where the second method of performing the alignment step S3 provides advantage over the first method of performing the alignment step S3

A pair of series of measurements for a known 800 base DNA sequence and its reverse complement were simulated on the basis of characteristics derived by experiment on a known type of measurement system 8 of the type shown in FIG. 2 and described above. As the two base sequences have a predetermined relationship and how the measurement sequences are constructed is controlled, then the true alignment mapping between the two measurement sequences is known. Many such simulations were performed, the first and second types of method described above to estimate the alignment mapping.

These estimated alignment mappings were then compared to the true alignment mapping by calculating a score representing the accuracy of the estimated alignment mappings were calculated using the following procedure.

Both the true alignment mapping and estimated alignment mapping are expressed as a list of Cartesian coordinates, in obvious notation: [(x1, y1), (x2, y2), (x2, y2), . . . , (xN, yN)], where N is the length of the alignment mapping, each xi refers to a measurement in the first sequence, and each yi refers to a measurement in the second sequence. In forming this expression of the alignment mapping, any gap in the sequence with a repeated value.

The score was derived as follows. For each entry in the list for the true alignment mapping, all entries in the list for the estimated alignment mapping with identical xi value were identified. Then the yi value from the list for the true alignment mapping were compared with the one or more yi values from list for the estimated alignment mapping. The maximum absolute difference between yi from the true list and yi from the estimated list was taken. The maximum of all such values across all entries in the true list was found. This gives a maximum vertical deviation of the two alignment mappings. Repeating the process with xi and yi interchanged in the former description yields a maximum horizontal difference. The score given to an estimated alignment mapping is then the maximum of the maximum horizontal and maximum vertical differences. Thus, a lower score indicates a more accurate estimate alignment.

The scores in respect of 100 pairs of series of measurements are plotted in FIG. 18 wherein the score for the first and second methods are plotted along the vertical and horizontal axes, respectively. In no case do the scores equal zero, but in a clear majority of cases, the second method produces a more accurate estimated alignment (i.e. a lower score) than the first method.

1. A method of generating an estimate of a target sequence of polymer units from plural series of measurements taken from respective measured sequences of polymer units in the same or different polymers, wherein the respective measured sequences correspond to the target sequence by comprising the target sequence or a sequence having a predetermined relationship with the target sequence, each measurement being dependent on a k-mer, being k polymer units of the respective sequence of polymer units, where k is an integer,
the method using a model in respect of each series of measurements that treats the measurements as observations of a series of k-mer states of different possible types, and comprises:
in respect of each transition between successive k-mer states in the series of k-mer states, transition weightings for possible transitions between the possible types of the k-mer states; and
in respect of each type of k-mer state, emission weightings for different measurements being observed when the k-mer state is of that type,
the method comprising:
deriving an estimated alignment mapping between the plural series of measurements; and
generating an estimate of the target sequence of polymer units from the plural series of measurements by applying the models in a manner that treats the types of k-mer state of each model as dimensions of a plural dimensional k-mer state and treats the measurements of each series as plural dimensional observations of those plural dimensional k-mer states, and that paths through the plural dimensional k-mer states are constrained using the derived alignment mapping between the plural series of measurements.

2. A method according to claim 1, wherein the step of deriving an estimated alignment mapping between the plural series of measurements is based on an application of the model in respect of each series of measurements to the measurements of that series

3. A method according to claim 2, wherein the step of deriving an estimated alignment mapping between the plural series of measurements comprises:
in respect of each series of measurements, generating a series of estimates of k-mer states by applying the model of the series of measurements to the series of measurements; and
deriving the estimated alignment mapping between the series of measurements by comparing the plural series of estimates of the k-mer states.

4. A method according to claim 3, wherein each estimate of a k-mer state comprises weightings for each possible type of k-mer state.

5. A method according to claim 4, wherein each generated series of estimates of k-mer states comprises an estimate of a k-mer state in respect of each measurement.

6. A method according to claim 3, wherein each estimate of a k-mer state comprises a discrete estimated k-mer state.

7. A method according to claim 6, wherein each generated series of estimates of the k-mer states comprises an estimated k-mer state in respect of each measurement.

8. A method according to claim 6, wherein
k is a plural integer, and
each generated series of estimates of k-mer states comprises an estimated k-mer state in respect of each k-mer in an underlying sequence that corresponds with an estimate of the measured sequence of polymer units, the estimated k-mer states in respect of each k-mer in the underlying sequence being mapped to the measurements.

9. A method according to claim 8, wherein the step of generating a series of estimates of k-mer states comprises:
generating an initial series of estimates of k-mer states in respect of each measurement by applying the model of the series of measurements to the series of measurements; and
analysing the initial series of estimates of k-mer states in respect of each measurement to derive the generated series of estimates of k-mer states as the estimated k-mer state in respect of each k-mer in said underlying sequence.

10. A method according to claim 3, wherein, in said step of deriving the estimated alignment mapping between the series of measurements, said comparing of the series of series of estimates of the k-mer states uses a substitution scoring function, in respect of possible alignment mappings that map each measurement of each series to a measurement in the other series or to a gap in the other series, that comprises a combination of (a) substitution scores, in respect of measurements in the plural series that that the possible alignment aligns together, representing the likelihood that the estimates of the k-mer states mapped to the aligned measurements are dependent on the same type of k-mer, and (b) a gap penalty, in respect of measurements in the plural series that the possible alignment aligns to a gap in the other series, representing the likelihood that the other series does not include a measurement of the same type of k-mer, and the possible alignment mapping that maximises the scoring function is derived as the estimated alignment mapping.

11. A method according to claim 10, wherein the possible alignment mapping that maximises the scoring function is derived as the estimated alignment mapping using a dynamic programming technique.

12. A method according to claim 10, wherein each estimate of a k-mer state comprises weightings for each possible type of k-mer state, and the substitution scores are derived from the weightings for each possible type of k-mer state.

13. A method according to claim 12, wherein the substitution scores are derived from a sum of the products of the weighting for each possible type of k-mer.

14. A method according to claim 10, wherein each estimate of a k-mer state comprises a discrete estimated k-mer state, and the substitution scores representing the likelihood that the discrete estimated k-mer states mapped to the aligned measurements are dependent on the same type of k-mer are derived from stored data representing such likelihoods for each possible combination of a type of k-mer state estimated from the first series of measurements and a type of k-mer state estimated from the second series of measurements.

15. A method according to claim 10, wherein the gap penalty is consistent with the likelihood predicted by model that the other series does not include measurement of the same k-mer.

16. A method according to claim 15, wherein the gap penalty is derived from a ratio of the likelihood predicted by model that a measurement is aligned to a gap in the other series to the likelihood predicted by model that a measurement is not aligned to a measurement in the other series.

17. A method according to claim 1, wherein the step of deriving an estimated alignment mapping between the plural series of measurements comprises:
in respect of a first one of the series of measurements, performing the steps of:
generating a series of estimated k-mer states by applying the model of the first series of measurements to the first series of measurements; and
generating a reference model that treats the series of measurements as observations of the generated series of estimated k-mer states, wherein the reference model comprises:
transition weightings for transitions between the k-mer states in the generated series of estimated k-mer states; and
in respect of each k-mer state, emission weightings for different measurements being taken when the k-mer state is observed; and
deriving an estimated alignment mapping between the plural series of measurements by applying the reference model to the other series of measurements.

18. A method according to claim 17, wherein the first series of measurements is of a higher data quality than the other series.

19. A method according to claim 1, wherein the estimated alignment mapping maps each measurement of each series to a measurement in the other series or to a gap in the other series.

20. A method according to claim 1, wherein one or both of the transition weightings and the emission weightings are probabilities.

21. A method according to claim 1, wherein the model is a Hidden Markov Model.

22. A method according to claim 1, wherein the model is stored in a memory.

23. A method according to claim 1, wherein the estimate of the target sequence of polymer units comprises weightings for different possible types of polymer unit.

24. A method according to claim 1, wherein said paths through the plural dimensional k-mer states are constrained to be within a predetermined distance from the derived alignment mapping between the plural series of measurements.

25. A method according to claim 1, wherein the respective sequences include sequences having a predetermined relationship with the target sequence of being complementary to the target sequence.

26. A method according to claim 1, wherein k is a plural integer.

27. A method according to claim 1, wherein said measurements are measurements taken during translocation of said same or different polymers through a nanopore.

28. A method according to claim 27, wherein the translocation of said same or different polymers through a nanopore is performed in a ratcheted manner.

29. A method according to claim 27, wherein the nanopore is a biological pore.

30. A method according to claim 1, wherein the polymer is a polynucleotide, and the polymer units are nucleotides.

31. A method according to claim 30, wherein the respective sequences comprise the target sequence and a sequence having a predetermined relationship with the target sequence of being complementary to the target sequence, the target sequence and the complementary sequence being linked by a bridging moiety.

32. A method according to claim 31, wherein
said measurements are measurements taken during translocation of said same or different polymers through a nanopore, and
the respective sequences are separated prior to translocation through the nanopore.

33. A method according to claim 31, wherein the respective sequences are separated by a polynucleotide binding protein.

34. A method according to claim 33, wherein the polynucleotide binding protein is a helicase.

35. A method according to claim 30, wherein the respective polynucleotide sequences are 2,000 nucleotides or greater, optionally 10,000 nucleotides or greater.

36. A method according to claim 1, wherein each series of measurements comprises a series of a predetermined plural number of measurements of different natures that are dependent on the same k-mer.

37. A method according to claim 1, wherein the measurements comprise one or more of current measurements, impedance measurements, tunnelling measurements, field effect transistor measurements and optical measurements.

38. A method according to claim 1, wherein
the method further comprises, before the step of deriving an estimated alignment mapping, deriving said plural series of measurements by:
receiving plural series of raw measurements from the respective measured sequences of polymer units in the same or different polymers, in which series of raw measurements groups of plural raw measurements are dependent on the same k-mer, without a priori knowledge of the number of measurements in the group, and
processing each series of raw measurements to identify successive groups of measurements and in respect of each identified group deriving a single measurement or plural measurements of different types to form said plural series of measurements.

39. A method according to claim 38, further comprising taking said plural series of raw measurements from the respective measured sequences of polymer units in the same or different polymers.

40. A method according to claim 1, wherein, in each of said plural series of measurements, groups of plural measurements are dependent on the same k-mer, without a priori knowledge of number of measurements in the group.

41. A method according to claim 40, further comprising taking said plural series of measurements from the respective measured sequences of polymer units in the same or different polymers.

42. (canceled)

43. (canceled)

44. An analysis system for generating an estimate of a target sequence of polymer units from plural series of measurements taken from respective measured sequences of polymer units in the same or different polymers, wherein the respective measured sequences correspond to the target sequence by comprising the target sequence or a sequence having a predetermined relationship with the target sequence, each measurement being dependent on a k-mer, being k polymer units of the respective sequence of polymer units, where k is an integer,
the analysis system comprising an analysis unit that is configured to use a model in respect of each series of measurements that treat the measurements as observations of a series of k-mer states of different possible types, and comprises:
in respect of each transition between successive k-mer states in the series of k-mer states, transition weightings for possible transitions between the possible types of the k-mer states; and
in respect of each type of k-mer state, emission weightings for different measurements being observed when the k-mer state is of that type,
the analysis unit being configured to perform the steps of:
deriving an estimated alignment mapping between the plural series of measurements based on an application of the model in respect of each series of measurements to the measurements of that series; and
generating an estimate of the target sequence of polymer units from the plural series of measurements by applying the models in a manner that treats the types of k-mer state of each model as dimensions of a plural dimensional k-mer state and treats the measurements of each series as plural dimensional observations of those plural dimensional k-mer states, and that paths through the plural dimensional k-mer states are constrained using the derived alignment mapping between the plural series of measurements.

45. A sequencing apparatus comprising:
a measurement system configured to make said measurements of a polymer; and
an analysis system according to claim 44.


SEQUENCE LISTING (9 sequences)

SEQ ID NO: 1 - DNA, 558 bp, Artificial Sequence
  MS-(B1)8 = MS-(D90N/D91N/D93N/D118R/D134R/E139K)8
atgggtctggataatgaactgagcctggtggacggtcaagatcgtaccctgacggtgcaacaatgggatacctttctgaatggcgtttttccgctggatcgtaatcgcctgacccgtgaatggtttcattccggtcgcgcaaaatatatcgtcgcaggcccgggtgctgacgaattcgaaggcacgctggaactgggttatcagattggctttccgtggtcactgggcgttggtatcaacttctcgtacaccacgccgaatattctgatcaacaatggtaacattaccgcaccgccgtttggcctgaacagcgtgattacgccgaacctgtttccgggtgttagcatctctgcccgtctgggcaatggtccgggcattcaagaagtggcaacctttagtgtgcgcgtttccggcgctaaaggcggtgtcgcggtgtctaacgcccacggtaccgttacgggcgcggccggcggtgtcctgctgcgtccgttcgcgcgcctgattgcctctaccggcgacagcgttacgacctatggcgaaccgtggaatatgaactaa

SEQ ID NO: 2 - protein, 184 aa, Artificial Sequence
  MS-(B1)8 = MS-(D90N/D91N/D93N/D118R/D134R/E139K)8
GLDNELSLVDGQDRTLTVQQWDTFLNGVFPLDRNRLTREWFHSGRAKYIVAGPGADEFEGTLELGYQIGFPWSLGVGINFSYTTPNILINNGNITAPPFGLNSVITPNLFPGVSISARLGNGPGIQEVATFSVRVSGAKGGVAVSNAHGTVTGAAGGVLLRPFARLIASTGDSVTTYGEPWNMN

SEQ ID NO: 3 - DNA, 558 bp, Artificial Sequence
  MS-(B2)8 = MS-(L88N/D90N/D91N/D93N/D118R/D134R/E139K)8
atgggtctggataatgaactgagcctggtggacggtcaagatcgtaccctgacggtgcaacaatgggatacctttctgaatggcgtttttccgctggatcgtaatcgcctgacccgtgaatggtttcattccggtcgcgcaaaatatatcgtcgcaggcccgggtgctgacgaattcgaaggcacgctggaactgggttatcagattggctttccgtggtcactgggcgttggtatcaacttctcgtacaccacgccgaatattaacatcaacaatggtaacattaccgcaccgccgtttggcctgaacagcgtgattacgccgaacctgtttccgggtgttagcatctctgcccgtctgggcaatggtccgggcattcaagaagtggcaacctttagtgtgcgcgtttccggcgctaaaggcggtgtcgcggtgtctaacgcccacggtaccgttacgggcgcggccggcggtgtcctgctgcgtccgttcgcgcgcctgattgcctctaccggcgacagcgttacgacctatggcgaaccgtggaatatgaactaa

SEQ ID NO: 4 - protein, 184 aa, Artificial Sequence
  MS-(B2)8 = MS-(L88N/D90N/D91N/D93N/D118R/D134R/E139K)8
GLDNELSLVDGQDRTLTVQQWDTFLNGVFPLDRNRLTREWFHSGRAKYIVAGPGADEFEGTLELGYQIGFPWSLGVGINFSYTTPNININNGNITAPPFGLNSVITPNLFPGVSISARLGNGPGIQEVATFSVRVSGAKGGVAVSNAHGTVTGAAGGVLLRPFARLIASTGDSVTTYGEPWNMN

SEQ ID NO: 5 - protein, 485 aa, Escherichia coli
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGSGHHHHHH

SEQ ID NO: 6 - protein, 268 aa, Escherichia coli
MKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRR

SEQ ID NO: 7 - protein, 666 aa, Thermus thermophilus
MRDRVRWRVLSLPPLAQWREVMAALEVGPEAALAYWHRGFRRKEDLDPPLALLPLKGLREAAALLEEALRQGKRIRVHGDYDADGLTGTAILVRGLAALGADVHPFIPHRLEEGYGVLMERVPEHLEASDLFLTVDCGITNHAELRELLENGVEVIVTDHHTPGKTPSPGLVVHPALTPDLKEKPTGAGVVFLLLWALHERLGLPPPLEYADLAAVGTIADVAPLWGWNRALVKEGLARIPASSWVGLRLLAEAVGYTGKAVEVAFRIAPRINAASRLGEAEKALRLLLTDDAAEAQALVGELHRLNARRQTLEEAMLRKLLPQADPEAKAIVLLDPEGHPGVMGIVASRILEATLRPVFLVAQGKGTVRSLAPISAVEALRSAEDLLLRYGGHKEAAGFAMDEALFPAFKARVEAYAARFPDPVREVALLDLLPEPGLLPQVFRELALLEPYGEGNPEPLFLLFGAPEEARRLGEGRHLAFRLKGVRVLAWKQGDLALPPEVEVAGLLSENAWNGHLAYEVQAVDLRKPEALEGGIAPFAYPLPLLEALARARLGEGVYVPEDNPEGLDYARKAGFRLLPPEEAGLWLGLPPRPVLGRRVEVALGREARARLSAPPVLHTPEARLKALVHRRLLFAYERRHPGLFSEALLAYWEVNRVQEPAGSP

SEQ ID NO: 8 - protein, 226 aa, Bacteriophage lambda
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 9 - protein, 608 aa, Bacteriophage phi-29
MKHMPRKMYSCAFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSAWSHPQFEKGGGSGGGSGGSAWSHPQFEK